(19) United States
(12) Patent Application Publication
(10) Pub. No.: US 2021/0251203 A1
(43) Pub. Date: Aug. 19, 2021

(54) POLYNUCLEOTIDE

(71) Applicant: IMPERIAL COLLEGE OF SCIENCE, TECHNOLOGY AND MEDICINE, London (GB)

(72) Inventors: Andrea Crisanti, London (US); Tony Nolan, Liverpool (GB); Andrew Hammond, London (GB)

(73) Assignee: IMPERIAL COLLEGE OF SCIENCE, TECHNOLOGY AND MEDICINE, London (GB)

(21) Appl. No.: 17/253,491

(22) PCT Filed: Jun. 21, 2019
(86) PCT No.: PCT/GB2019/051749

§ 371 Date: Dec. 17, 2020

(30) Foreign Application Priority Data
Jun. 22, 2018 (GB) 1810256.6

(51) Int. Cl. A01K 67/033; C12N 15/85

(52) U.S. Cl. CPC A01K 67/0339; C12N 15/8509; C12N 2830/008; A01K 2227/706; A01K 2217/15; A01K 2217/206; A01K 2217/072

(57) ABSTRACT

The invention relates to polynucleotides, and in particular to novel polynucleotides which represent promoter sequences. The invention is especially concerned with novel promoters for use in germline expression, in that they are substantially operative in only germline cells. In particular, the promoters initiate transcription of genes in the germline cells of an arthropod, and can be used in a gene drive. The invention is also concerned with vectors and gene drive constructs comprising the polynucleotides of the invention. The invention is also concerned with methods of producing arthropods comprising vectors containing such promoters.

The invention relates to polynucleotides, and in particular to novel polynucleotides which represent promoter sequences. The invention is especially concerned with novel promoters for use in germline expression, in that they are substantially operative in only germline cells. In particular, the promoters initiate transcription of genes in the germline cells of an arthropod, and can be used in a gene drive. The invention is also concerned with vectors and gene drive constructs comprising the polynucleotides of the invention. The invention is also concerned with methods of producing arthropods comprising vectors containing such promoters.

A gene drive is a genetic engineering approach that can propagate a particular suite of genes throughout a target population. Gene drives have been proposed to provide a powerful and effective means of genetically modifying specific populations and even entire species. For example, applications of gene drive include exterminating insects that carry pathogens (e.g. mosquitoes that transmit malaria, dengue and zika pathogens), controlling invasive species, or eliminating herbicide or pesticide resistance.

CRISPR-Cas9 nucleases have recently been employed in gene drive systems to target endogenous sequences of the human malaria vector Anopheles gambiae and Anopheles stephensi with the objective to develop genetic vector control measures^{1, 2}. These initial proof-of-principle experiments have demonstrated the potential of gene drive approaches and translated a theoretical hypothesis into a powerful genetic tool potentially capable of modifying the genetic makeup of a species and changing its evolutionary destiny either by suppressing its reproductive capability or permanently modifying the outcome of the mosquito interaction with the malaria parasites they transmit.

The recent proof-of-principle demonstration of gene drive applications for vector control of human malaria mosquitoes have translated a theoretical hypothesis into a most powerful genetic tool potentially capable of modifying the genetic makeup of a species and changing its evolutionary destiny. The wide range of applicability of gene drive technology to control insect pests as well as many invasive species including rodents has generated a worldwide scientific effort aimed at developing more effective and safer version of the technology. A key factor in the development of effective and safe gene drive technology is the availability of regulatory promoter sequences to restrict the expression of the drive nucleases exclusively in the male and female mosquito germline at the time of meiosis to avoid unwanted toxic effects on somatic tissues and at the same time minimise the generation of drive-resistant mutants.

Tissue-specific promoters are a powerful tool in restricting the expression of a transgene to specific cell or tissue types. Use of tissue-specific promoters can restrict unwanted transgene expression, as well as facilitate persistent transgene expression. Therefore, novel promoter sequences that are operative in a given tissue are highly desired.

As described in the Examples, the inventors have identified three novel regulatory sequences (also called “promoters”), which are referred to herein as nanos (nos), zero population (zpg), and exuperentia (exu), each of which regulates the expression of transgenes in host germline cells, and which can therefore be used in gene drive approaches, for example in mosquitoes. These sequences that express transgenes in the mosquito germline overcome a major roadblock in current gene drive design due to the difficulty to adequately restrict expression of Cas9 endonuclease to the germline. The leaky expression of nuclease activity in somatic tissue represents a major source of fitness reduction and of generation of functional drive-resistant nuclease target sequences. To this end, the inventors have validated and characterised the use of the three novel regulatory DNA sequences that are able to generate improved germline-restricted transgene expression in the malaria mosquito Anopheles gambiae, and other closely related species.

These three regulatory sequences, named “zpg”, “nos” and “exu”, each consist of two sequences of approximately 2 kb and 0.5-1 kb of DNA, and were isolated from the Anopheles gambiae genome (regulatory sequences from the genes zpg/zero population growth—AGAP006241, nos/nanos—AGAP006098, and exu/exuperantia—AGAP007365).

Accordingly, in a first aspect of the invention, there is provided an isolated polynucleotide comprising a nucleic acid sequence substantially as set out in any one of SEQ ID No: 1, 2 or 3, or a variant or fragment thereof having at least 50% sequence identity with SEQ ID No: 1, 2 or 3.

Advantageously, the inventors have shown that the polynucleotides of the first aspect behave as promoters which drive tissue-specific gene expression in the germline cells only. Accordingly, as described in the Examples, in a gene drive approach, use of the promoters of the invention restricts expression of Cas9 endonuclease to the germline, and therefore mitigates and prevents the emergence of resistant alleles by reducing the embryonic source of end-joining mutations.

In one preferred embodiment, the polynucleotide sequence may be referred to as “zero population” or “zpg”, which is provided herein as SEQ ID No: 1, as follows:

[SEQ ID No: 1]
cagcgctggcggtggggacagctccggctgtggctgttcttgCgagtcC
tcttcctgcggcacatccctctcgtcgaccagttcagtttgctgagcgt
aagcctgctgctgttcgtcctgcatcatcgggaccatttgtaTgggcca
tccgccaccaccaccatcaccaccgccgtccatttctaggggcataccc
atcagcatctccgcgggcgccattggcggtggtgccaaggtgccattcg
tttgttgctgaaagcaaaagaaagcaaattagtgttgtttctgctgcac
acgataAttttcgtttcttgccgctagacacaaacaacactgcatctgg
agggagaaatttgacgcctagctgtataacttacctcaaagttattgtc
catcgtggtataatggacctaccgagcccggttacactacacaaagcaa
gattatgcgacaaaatcacagcgaaaactagtaattttcatctatcgaa
agcggccgagcagagagttgtttggtattgcaacttgacattctgctgC
gggataaaccgcgacgggctaccatggcgcacctgtcagatggctgtca
aatttggcccggtttgcgatatggagtgggtgaaattatatcccactcg
ctgatcgtgaaaatagacacctgaaaacaataattgttgtgttaatttt
acattttgaagaacagcacaagttttgctgacaatatttaattacgttt
cgttatcaacggcacggaaagattatctcgctgattatccctctcgctc
tctctgtctatcatgtcctggtcgttctcgcgtcaccccggataatcga
gagacgccatttttaatttgaactactacaccgacaagcatgccgtgag
ctctttcaagttcttctgtccgaccaaagaaacagagaataccgcccgg
acagtgcccggagtgatcgatccatagaaaatcgcccatcatgtgccac
tgaGgcgaaccggcgtagcttgttccgaatttccaagtgcttccccgta
acatccgcatataacaaAcagcccaacaacaaatacagcatcgag

Accordingly, preferably the polynucleotide comprises or consists of a nucleic acid sequence substantially as set out in SEQ ID No: 1, or a variant or fragment thereof.

In another preferred embodiment, the polynucleotide sequence may be referred to as “nanos” or “nos”, and is provided herein as SEQ ID No: 2, as follows:

[SEQ ID No: 2]
gtgaacttccatggaattacgtgctttttcggaatggagttgggctggt
gaaaaacacctatcagcaccgcacttttcccccggcatttcaggttata
cgcagagacagagactaaatattcacccattcatcacgcactaacttcg
caatagattgatattccaaaactttcttcacctttgccgagttggattc
tggattctgagactgtaaaaagtcgtacgagctatcatagggtgtaaaa
cggaaaacaaacaaacgtttaatggactgctccaactgtaatcgcttca
cgcaaacaaacacacacgcgctgggagcgttcctggcgtcacctttgca
cgatgaaaactgtagcaaaactcgcacgaccgaaggctctccgtccctg
ctggtgtgtgtttttttcttttctgcagcaaaattagaaaacatcatca
tttgacgaaaacgtcaactgcgcgagcagagtgaccagaaataccgatg
tatctgtatagtagaacgtcggttatccgggggcggattaaccgtgcgc
acaaccagttttttgtgcagctttgtagtgtctagtggtattttcgaaa
ttcatttttgttcattaacagttgttaaacctatagttattgattaaaa
taatattctactaacgattaaccgatggattcaaagtgaataaattatg
aaactagtgatttttttaaatttttatatgaatttgacatttcttggac
cattatcatcttggtctcgagctgcccgaataatcgacgttctactgta
ttcctaccgattttttatatgcctaccgacacacaggtgggccccctaa
aactaccgatttttaatttatcctaccgaaaatcacagattgtttcata
atacagaccaaaaagtcatgtaaccatttcccaaatcacttaatgtatt
aaactccatatggaaatcgctagcaaccagaaccagaagttcaacagag
acaaccaatttccgtgtatgtacttcatgagatgagattggacgcgctg
gtaaaattttatatgggatttgacagataatgtaaggcgtgcgattttt
ttcatacgatggaatcaattcaagagtcaattgtgcaggatttatagaa
acaatctcttatttatgttttgttatcgttacagttacagccctgtcct
aagcggccgcgtgaaggcccaaaaaaaagggagtccccaacgctcagta
gcaaatgtgcttctctatcattcgttgggttagaaaagcctcatgtgac
ttctatgaacaaaatctaaactatctcctttaaatagagaatggatgta
ttttttcgtgccactgaactttcgttgggaagattagatacctctccct
ccccccccctccctttcaacacttcaaaacctaccgaaaactaccgata
caatttgatgtacctaccgaagaccgccaaaataatctggccacactgg
ctagatctgatgttttgaaacatcgccaaattttactaaataatgcact
tgcgcgttggtgaagctgcacttaaacagattagttgaattacgctttc
tgaaatgtttttattaaacacttgttttttttaatacttcaatttaaag
ctacttcttggaatgataattctacccaaaaccaaaaccactttacaaa
gagtgtgtggttggtgatcgcgccggctactgcgacctgtggtcatcgc
tcatctcacgcacacatacgcacacatctgtcatttgaaaagctgcaca
caatcgtgtgttgtgcaaaaaaccgttcgcgcacaaacagttcgcacat
gtttgcaagccgtgcagcaaagggcttttgatggtgatccgcagtgttt
ggtcagctttttaatgtgttttcgcttaatcgcttttgtttgtgtaatg
ttttgtcggaataatttttatgcgtcgttacaaatgaaatgtacaatcc
tgcgatgctagtgtaaaacattgctaattcccggtaagaacgttcatta
cgctcggatatcatcttacgaagcgTGTGTATGTGCGCTAGTACATTGA
CCTTTAAAGTgatccttttgttctagaaagcaag

Accordingly, preferably the polynucleotide sequence comprises or consists of a nucleic acid sequence substantially as set out in SEQ ID No: 2, or a variant or fragment thereof.

In yet another preferred embodiment, the second promoter sequence may be referred to as “exuperantia” or “exu”, and is provided herein as SEQ ID No: 3, as follows:

[SEQ ID No: 3]
ggaaggtgattgcgattccatgttgatgccaatatatgatgattttgtt
gcatattaatagttgttgttatgttttattcaaatttcaaagataattt
actttacattacagttagtgagcatattatctactacataaacacatag
atCaaactggtttacataaattcaaaaagtttgGattaaAatcgcagca
attggttatgaaaaaatatgtgCAtaacgtaaatatcaagtaaattttt
gcattgcatatttatagaCtcctgttacaatttcggaaaaatgaaaaat
gttaattaatcaaagaagaaaaaacaaagAaattaaatcattaggtAgc
acaaccacaagtacatatttttatggcatgaatattccTctacactaac
atattttatagcaattctattgatcgccttaGtatagcGgaattaccag
aacggcactatagttgtctctgtttggcacacgcaatcatttttcatcc
cagggttgccatagcagtttggcgacggtcacgtagcatgcgaaggatt
tcgTtcgcacaggatcacttttattctaacgtttgaagaagGcacatct
cagtgcaagcgctctggaagctgcttttaccgaacgaactaacttttca
agtaacctcaaaaacttgtctctaacgacaccacgtgctatccgcgagt
tTcatttcccgtgcaaagttccccgatttagctatcattcgtgaacatt
tcgtagtgcctctaccctcaggtaagaccattcgaGgtttaccaagttt
tgtgcaaagaaCGTGCacagtaattttCgttctggtgaaaccttctctt
gtgtagcttgtacaaa

Accordingly, preferably the polynucleotide sequence comprises or consists of a nucleic acid sequence substantially as set out in SEQ ID No: 3, or a variant or fragment thereof.

Preferably, the polynucleotide initiates gene expression of a coding sequence operatively connected thereto in the germline cells only. Preferably, the polynucleotide is operative in an arthropod cell. Preferably, the polynucleotide sequence is a promoter sequence that is substantially operative in only germline cells of an arthropod. More preferably, the polynucleotide is a promoter sequence which is substantially operative in the male and female mosquito gonad cells at the time of meiosis.

By “substantially operative”, it would be recognised by a person skilled in the art that there may be some degree of “leakiness” of the gene expression controlled by the polynucleotide of the invention, such that it may be operative in other tissues (e.g. of an arthropod). For example, preferably at least 60%, 70%, 80%, 90%, 95%, 96%, 97%, 98% or 99% of gene expression initiated by the polynucleotide of the invention is limited to the cell and or tissue of interest, i.e. the germline cells. Preferably, the sequences may only be operative in the desired cells.

Suitable arthropods for which the polynucleotide of the invention may operate include insects, arachnids, myriapods or crustaceans. Preferably, the arthropod is an insect. Preferably, the arthropod, and most preferably the insect, is a disease-carrying vector or pest (e.g. agricultural pest), which can infect, cause harm to, or kill, an animal or plant of agricultural value, for example, Anopheline species, Aedes species (as disease vectors), Ceratitis capitat, or Drosophila species (as an agricultural pest).

Preferably, the insect is a mosquito. Preferably, the mosquito is of the subfamily Anophelinae. Preferably, the mosquito is selected from a group consisting of: Anopheles gambiaes; Anopheles coluzzi; Anopheles merus; Anopheles arabiensis; Anopheles quadriannulatus; Anopheles stephensi; Anopheles arabiensis; Anopheles funestus; and Anopheles melas.

Most preferably, the mosquito is Anopheles gambiae.

Preferably, the polynucleotide is disposed in an expression cassette. Preferably, the expression cassette comprises the polynucleotide of the first aspect (i.e. the promoter), an open reading frame, and optionally a 3′ untranslated region, which may comprise a polyadenylation site.

Thus, in a second aspect, there is provided an expression cassette comprising the polynucleotide according to the first aspect operably linked to a transgene.

The cassette may further comprise a 3′ untranslated region involved with regulating expression of the transgene Preferably, the 3′ untranslated region comprises a 3′-polyadenylation sequence.

“Transgene” can refer to any exogenous nucleic acid sequence, in particular one for which germline expression is required. Preferably, the transgene is a nucleic acid that modifies the genome of the arthropod when expressed in its cells.

Preferably, the transgene is selected from a group consisting of: a CRISPR nuclease, Zinc finger nuclease, TALEN derived nucleases, a piggyback transposase, Cre recombinase, or a φC31 integrase.

Preferably, the transgene encodes a CRISPR nuclease, more preferably Cpf1 or Cas9. Most preferably, the transgene encodes Cas9.

The polynucleotide of the invention is preferably disposed in a recombinant vector, for example a recombinant vector for delivery into a host cell of interest.

Accordingly, in a third aspect, there is provided a recombinant vector comprising the polynucleotide according to the first aspect, or the expression cassette according to the second aspect.

The vector may for example be a plasmid, cosmid, phage and/or viral vector. Such recombinant vectors are highly useful in delivering the transgene to a host cell. Recombinant vectors may also include other functional elements. For example, they may further comprise a variety of other functional elements including a suitable regulatory sequence for controlling transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. The cassette or vector may also comprise a terminator, such as the Beta globin, SV40 polyadenylation sequences or synthetic polyadenylation sequences. The recombinant vector may also further comprise a regulator or enhancer to control expression of the nucleic acid as required. Tissue specific enhancer elements may be used in addition to the polynucleotide sequences described herein to further regulate expression of the nucleic acid in germ cells, preferably of an arthropod.

The vector may also comprise DNA coding for a gene that may be used as a selectable marker in the cloning process, i.e. to enable selection of host cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the polynucleotide and transgene. The cassette or vector may also further comprise other DNA involved with regulating expression of the transgene.

Purified vector may be inserted directly into a host cell by suitable means, e.g. direct endocytotic uptake. The vector may be introduced directly into cells of a host arthropod (e.g. a mosquito) by transfection, infection, electroporation, microinjection, cell fusion, protoplast fusion or ballistic bombardment. Alternatively, vectors of the invention may be introduced directly into a host cell using a particle gun.

The nucleic acid molecule may (but not necessarily) be one, which becomes incorporated in the DNA of cells of the subject being treated. Undifferentiated cells may be stably transformed leading to the production of genetically modified daughter cells (in which case regulation of expression in the subject may be required e.g. with specific transcription factors or gene activators). Alternatively, the vector may be designed to favour unstable or transient transformation of differentiated cells in the subject being treated. When this is the case, regulation of expression may be less important because expression of the DNA molecule will stop when the transformed cells die or stop expressing the protein.

The polynucleotide, expression cassette or vector may be transferred to the cells of the host by transfection, infection, microinjection, cell fusion, protoplast fusion or ballistic bombardment. For example, transfer may be by ballistic transfection with coated gold particles, liposomes containing the nucleic acid molecule, viral vectors (e.g. adenovirus) and means of providing direct nucleic acid uptake (e.g. endocytosis) by application of the nucleic acid molecule directly.

In a fourth aspect, there is provided a host cell comprising the expression cassette of the second aspect, or the recombinant vector of the third aspect.

The host cell may be prokaryotic. Preferably, however, the host cell is eukaryotic. Preferably, the host cell is an arthropod cell, as described in relation to the first aspect. Preferably, the arthropod cell is an insect cell. Preferably, the arthropod cell, and most preferably the insect cell, is a cell of a disease-carrying vector or pest (e.g. agricultural pest), which can infect, cause harm to, or kill, an animal or plant of agricultural value, for example, Anopheline species, Aedes species (as disease vectors), Ceratitis capitat, or Drosophila species (as an agricultural pest).

Preferably, the insect cell is a mosquito cell. Preferably, the mosquito is of the subfamily Anophelinae. Preferably, the mosquito cell is selected from a group consisting of: Anopheles gambiaes; Anopheles coluzzi; Anopheles merus; Anopheles arabiensis; Anopheles quadriannulatus; and Anopheles melas. Most preferably, the mosquito cell is an Anopheles gambiae cell.

In a fifth aspect, there is provided a method of producing a genetically modified host cell comprising introducing, into a host cell, the expression cassette of the second aspect, or the vector according to the third aspect.

Preferably, the host cell is as described in the fourth aspect.

In a sixth aspect, there is provided a genetically modified host cell obtained or obtainable by the method of the fifth aspect.

Preferably, the host cell is as described in the fourth aspect.

The polynucleotides of the present invention are particularly useful for driving germline specific expression of gene drive constructs.

Advantageously, the regulatory sequences of zpg (SEQ ID No: 1), nos (SEQ ID No: 2) and exu (SEQ ID No:3) described herein offer a clear advantage over and above the best system that is currently available (i.e. the vasa2 promoter, which may also be known as vas2) used for germline nuclease expression in gene drives designed for the malaria mosquito, showing: (1) high rates of biased transmission into the offspring of both male and female mosquitoes, (2) substantially reduced fitness cost, (3) reduced end-joining mutations that are the major cause of resistance to gene drive, and (4) vastly improved spread in caged experiments in terms of speed, persistence and maximum frequency of the drive.

Surprisingly, gene drives based upon the polynucleotide sequences disclosed herein are far superior to all previously tested gene drives and could be used for both population replacement and population suppression strategies. The improvements in gene drive efficacy can be attributed to vast improvements in spatio-temporal regulation of nuclease expression, preferably Cas 9, which is brought about by the use of these novel regulatory sequences, specifically an improvement in restriction to the germline.

To illustrate the magnitude of improvement, the inventors observed a relative fitness in females of more than 80% compared to only 7% using the vasa2 promoter. The ultimate goal of gene drive technology is to modify entire populations when starting from low initial release frequency, using identical methods to previously published research the inventors have observed the first ever spread to >99% of individuals in a caged population using the zpg promoter, compared to a maximum frequency of 80% in the previous best tested gene drive based upon the vasa2 promoter. The inventors have demonstrated this spread when releasing from 50% initial frequency (mirroring previous research) and also from 10% initial frequency that is more relevant to vector control. The improved activity can be attributed entirely to the use of improved germline promoters because the gene drives were otherwise identical and the observed improvements in spread are predicted by mathematical models based upon observed characteristics of the transgenic lines based upon these promoters. Surprisingly, the inventors have demonstrated that gene drives built using these promoters require no further improvement to invade entire mosquito populations and meet the requirements for a gene drive system aimed at population replacement.

Accordingly, in an seventh aspect of the invention, there is provided a gene drive genetic construct comprising the polynucleotide according to the first aspect, the expression cassette of the second aspect, or the vector according to the third aspect.

The skilled person will appreciate that the gene drive construct of the invention may relate to a construct comprising one or more genetic elements that biases its inheritance above that of Mendelian genetics, and thus increases in its frequency within a population over a number of generations.

Preferably, the polynucleotide sequence substantially restricts the activity of the gene drive genetic construct for germline expression of the construct in an arthropod.

Preferably, the arthropod is as described in the first aspect.

Preferably, the polynucleotide substantially restricts activity of the gene drive genetic construct to germline cells of an arthropod. More preferably, the polynucleotide substantially restricts activity of the gene drive genetic construct to the male and female mosquito gonads at the time of meiosis.

Preferably, the gene drive construct targets a gene sequence associated with a female arthropod's reproductive capacity, such that the targeting of the gene sequence with the gene drive construct results in suppression of a female's reproductive capacity. The skilled person would understand that suppression of a female's reproductive capacity may relate to a reduced ability to procreate, or complete sterility.

Alternatively, the promoter sequence may be used to spread genes that confer resistance to pathogen ability to colonize the vector and hence produce vectors that are disease immune.

It will be appreciated that suppression of a female's reproductive capacity can relate to a reduced ability of the female of the specific to procreate, or complete sterility of the female. Preferably, the reproductive capacity of the female homozygous for the construct is reduced by at least 5%, 10%, 20% or 30% compared to the corresponding wild-type female. More preferably, the reproductive capacity of the female homozygous for the construct is reduced by at least 40%, 50% or 60% compared to the corresponding wild-type female. Most preferably, the reproductive capacity of the female homozygous for the construct is reduced by at least 70%, 80% or 90% compared to the corresponding wild-type female. Most preferably, suppression of a female's reproductive results in complete sterility of the female.

The concept of gene drive genetic constructs is known to those skilled in the art. Preferably, the gene drive genetic construct is a nuclease-based genetic construct. The gene drive genetic construct may be selected from a group consisting of: a transcription activator-like effector nuclease (TALEN) genetic construct; Zinc finger nuclease (ZFN) genetic construct; and a CRISPR-based gene drive genetic construct. Preferably, the genetic construct is a CRISPR-based gene drive construct, most preferably a CRISPR-Cpf1-based or CRISPR-Cas9-based gene drive genetic construct.

Preferably, the targeting of a gene by the gene drive genetic gene drive construct results in:
i) unisexual sterility;
ii) bisexual sterility; or
iii) bisexual lethality.

Preferably, the gene to be targeted by the genetic gene drive construct is a female fertility gene from Anopheles gambiae.

Preferably, the gene to be targeted by the genetic gene drive construct is selected from a group consisting of: AGAP005958, AGAP007280, AGAP0011377 and AGAP004050, or an orthologue thereof.

Most preferably, the gene to be targeted by the genetic gene drive construct is the doublesex (dsx) gene. In one embodiment, the doublesex gene is from Anopheles gambiae (referred to as AGAP004050). Advantageously, this doublesex gene is highly conserved with strict sequence constraints, and so presents a preferred target gene.

Accordingly, in an embodiment in which the genetic construct is a CRISPR-based gene drive genetic construct, the genetic construct further comprises a first polynucleotide sequence encoding a polynucleotide sequence that is capable of hybridising to the sequence of a gene which is to be targeted. Preferably, the first polynucleotide sequence is a guide RNA.

Preferably, the CRISPR-based gene drive genetic construct further comprises a second polynucleotide sequence encoding a CRISPR nuclease, preferably a Cpf1 or Cas9 nuclease, most preferably a Cas9 nuclease. The sequences of the preferred nuclease and encoding nucleotides are known in the art. Preferably, the second polynucleotide sequence encoding the nuclease is disposed 5′ of the first nucleotide sequence encoding a polynucleotide sequence that is capable of hybridising to the sequence of a gene which is to be targeted.

Preferably, the polynucleotide sequence substantially as set out in any one of SEQ ID Nos: 1, 2 or 3, or a fragment or variant thereof is operably linked to the second nucleotide sequence and a second promoter sequence is operably linked to the first nucleotide sequence.

The second promoter sequence may be any promoter sequence that is suitable for expression in an arthropod, and which would be known to those skilled in the art.

In one embodiment, the first nucleotide sequence may be produced by self-cleaving RNA elements, such as tRNA, Cys4 or ribozyme sequences, such as the hammerhead ribozyme and hepatitis delta virus ribozyme. Such methods are known to those skilled in the art.

In embodiments where the first nucleotide sequence is produced by self-cleaving RNA elements, the second promoter sequence may be the polynucleotide sequence substantially as set out in any one of SEQ ID Nos: 1, 2 or 3, or a fragment or variant thereof.

Preferably, the second promoter is a polymerase III promoter, preferably a polymerase III promoter which does not add a 5′cap or a 3′polyA tail. More preferably, the promoter is U6

The skilled person would understand that the polynucleotide sequence that is capable of hybridising to the to the sequence of a gene which is to be targeted may further comprise a CRISPR nuclease binding sequence, preferably a Cpf1 or Cas9 nuclease binding sequence, and most preferably a Cas9 nuclease binding sequence.

Preferably, when transcribed, the first polynucleotide sequence, which hybridises to the intron-exon boundary, targets the nuclease to the intron-exon boundary of the doublesex gene, and the nuclease cleaves the doublesex gene at the intron-exon boundary, such that the gene drive construct is integrated into the disrupted intron-exon boundary via homology-directed repair. The skilled person would understand that once the gene drive is inserted into the genome of the arthropod, it will use the natural homology found at the site in which it is inserted in the genome.

It will be appreciated that the gRNA is not necessarily directed against the doublesex gene, and the promoters of the invention can be used to develop drive targeting different gene for either population suppression or population replacement.

The gene drive genetic construct may be inserted directly into a host cell by suitable means, e.g. direct endocytotic uptake. The construct may be introduced directly into cells of a host subject (e.g. a mosquito) by transfection, infection, electroporation, microinjection, cell fusion, protoplast fusion or ballistic bombardment. Alternatively, constructs of the invention may be introduced directly into a host cell using a particle gun.

Preferably, the construct is introduced into a host cell by microinjection of arthropod embryos, preferably insect embryos most preferably mosquito embryos. Preferably, the mosquito is of the subfamily Anophelinae, and more preferably the mosquito is any one of: Anopheles gambiae, Anopheles coluzzi, Anopheles stephensi, Anopheles arabiensis, Anopheles melas and Anopheles funestus. Most preferably, the mosquito is Anopheles gambiae.

Thus, the inventors has developed regulatory promoter sequences to restrict the expression of the drive nucleases exclusively in the male and female mosquito gonads at the time of meiosis to avoid unwanted toxic effects on somatic tissues and at the same time minimise the generation of drive resistant mutants.

Advantageously, the inventors have used these sequences to express Cas9 endonuclease in the context of a gene drive in the malaria mosquito and demonstrate surprising superiority over the previously used best alternative, the vasa2 promoter (https://www.nature.com/articles/nbt3439).

The technical effect of these novel promoter sequences includes: 1) improved transmission into the offspring of female mosquitoes resulting in higher net transmission of the gene drive, 2) reduced fitness costs, 3) reduced generation of end-joining mutations that can cause resistance to gene drive, and 4) improved spread in caged experiments in terms of speed, persistence and maximum frequency of the drive.

Most importantly, the inventors demonstrate that gene drives based upon the Zero Population Growth (zpg) promoter can spread through an entire population of mosquitoes in a demonstration that is both unprecedented and the ultimate goal of a gene drive system. Using the regulatory sequences described herein, the inventors have demonstrated that it is now possible to build gene drives aimed at population replacement in the malaria mosquito. The inventors have also demonstrated successful use of these sequences for mosquito transformation using CRISPR-based homology directed repair and, while not wishing to be bound to any particular theory, hypothesise that these regulatory sequences will also be useful for other methods of mosquito transformation (e.g. using these regulatory sequences to express piggyback transposase, Cre recombinase or φC31 integrase) and mosquito transgenesis more generally.

The inventors used bioinformatics analysis to identify these sequences, the translational start and stop sites, and untranslated regions that could further restrict expression of maternally or paternally derived transcripts by restricting translation to the germline (thought to be a major drawback of the vasa2 promoter). Importantly, nuclease deposition into the embryo is thought to be a major source of resistance to gene drive (http://journals.plos.org/plosgenetics/article?id=10.1371/journal.pgen.1007039) and the inventors designed these regulatory sequences to minimise this activity.

These sequences were not obvious choices for use in a gene drive. Indeed, “nos (also known as nanos) zpg and exu are believed to be inadequate for bi-sex gene drive expression in Anopheles gambiae because it was thought to be female-specific” (https://www.ncbi.nlm.nih.gov/pmc/articles/PMC43210324/, https://www.ncbi.nlm.nih.gov/pmc/articles/PMC43210324/ & https://www.sciencedirect.com/science/article/pii/S0965174805000603?via %3Dihub).

Prior to this work, another research group published the use of similarly named regulatory sequences isolated from the exuperantia gene of an unrelated mosquito species, Aedes aegypti (https://www.nature.com/articles/srep03954). These sequences were used to drive germline transgene mosquito in this species, the yellow fever mosquito Aedes aegypti. However, they bear no resemblance to the presently disclosed sequences which were isolated from Anopheles gambiae (24.4% sequence identity compared to 25% that would be predicted by chance alone) and have not been shown to work in the malaria mosquito, either for transgene expression or use in a gene drive.

The gene drive construct may for example be a plasmid, cosmid or phage and/or be a viral vector. Such recombinant vectors are highly useful in the delivery systems of the invention for transforming cells. The nucleic acid sequence may preferably be a DNA sequence. The gene drive construct may further comprise a variety of other functional elements including a suitable regulatory sequence for controlling expression of the genetic gene drive construct upon introduction of the construct in a host cell. The construct may further comprise a regulator or enhancer to control expression of the elements of the constructs required. Tissue specific enhancer elements, for example promoter sequences, may be used to further regulate expression of the construct in germ cells of an arthropod.

In an eighth aspect of the invention, there is provided a method of producing a genetically modified arthropod comprising introducing, into an arthropod gene, a gene drive genetic construct according to the seventh aspect.

Preferably, the arthropod is as defined in the first aspect.

The gene drive genetic construct may be introduced directly into an arthropod host cell, preferably an arthropod host cell present in an arthropod embryo, by suitable means, e.g. direct endocytotic uptake. The construct may be introduced directly into cells of a host arthropod (e.g. a mosquito) by transfection, infection, electroporation, microinjection, cell fusion, protoplast fusion or ballistic bombardment. Alternatively, constructs of the invention may be introduced directly into a host cell using a particle gun.

Preferably, the construct is introduced into a host cell by microinjection of arthropod embryos, preferably an insect embryo and most preferably mosquito embryos.

Preferably, the gene drive genetic construct is introduced by microinjection into freshly laid eggs, within 2 hours of deposition, using standard methods in the art. More preferably, the gene drive genetic construct is introduced into an arthropod embryo at the start of melanisation, which the skilled person would understand takes place within 30 minutes after egg laying.

Preferably, the mosquito is of the subfamily Anophelinae. Preferably, the mosquito is selected from a group consisting of: Anopheles gambiaes; Anopheles coluzzi; Anopheles merus; Anopheles arabiensis; Anopheles quadriannulatus; Anopheles stephensi; Anopheles arabiensis; Anopheles funestus; and Anopheles melas.

In a ninth aspect of the invention, there is provided a genetically modified arthropod obtained or obtainable by the method of the eighth aspect.

Preferably, the arthropod is as defined in the first aspect.

In a tenth aspect of the invention, there is provided a genetically modified arthropod comprising a gene drive genetic construct of the seventh aspect.

Preferably, the arthropod is as defined in the first aspect.

In a eleventh aspect of the invention, there is provided a method of suppressing a wild-type arthropod population, comprising breeding a genetically modified arthropod comprising gene drive construct capable of disrupting a gene associated with female reproductive capacity, with a wild type population of the arthropod, wherein the gene drive construct comprises the isolated polynucleotide of the first aspect, the expression cassette of the second aspect or the vector according to the third aspect.

Preferably, the arthropod is as defined in the first aspect. Preferably, the gene drive genetic construct is as defined in the seventh aspect.

In a twelfth aspect the invention, there is provided the use of a gene drive genetic construct comprising a polynucleotide sequence of the first aspect, the expression cassette of the second aspect or the vector according to the third aspect, to suppress a wild-type arthropod population.

Preferably, the arthropod is as defined in the first aspect. Preferably, the gene drive genetic construct is as defined in the seventh aspect.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-90 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3×sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 1 to 90.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

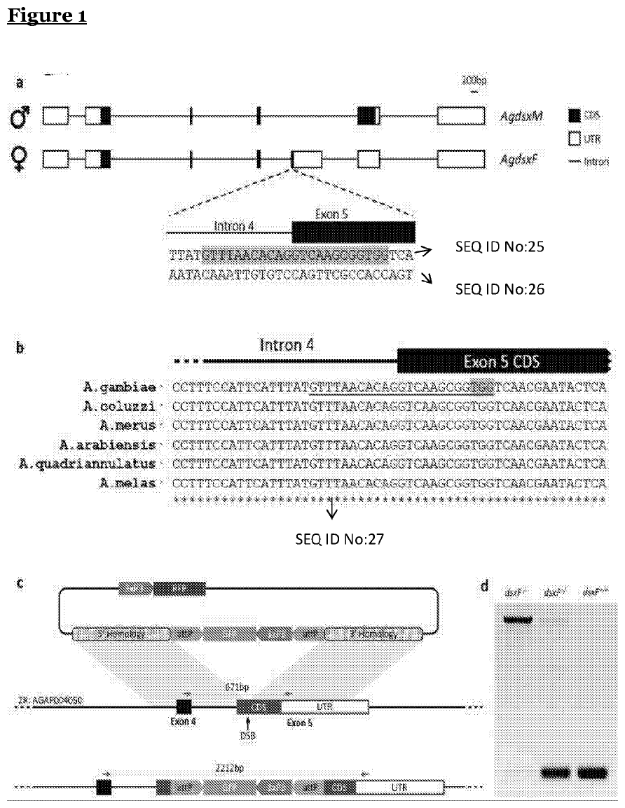

FIG. 1 shows targeting the female-specific isoform of doublesex. (a) Schematic representation of the male- and female-specific dsx transcripts and the gRNA sequence used to target the gene (shaded in grey). The gRNA spans the intron 4-exon 5 boundary. The PAM of the gRNA is highlighted in light grey. The scale bar indicates a 200 bp fragment. Coding regions of exons are shaded in black, non-coding regions in white. Introns are not drawn to scale. (b) Sequence alignment of the dsx intron 4-exon 5 boundary in 6 of the species from the Anopheles gambiae complex. The sequence is highly conserved within the complex suggesting tight functional constraint at this region of the dsx gene. The gRNA used to target the gene is underlined and the PAM is highlighted in grey. (c) Schematic representation of the HDR knockout construct specifically recognising exon 5 and the corresponding target locus. (d) Diagnostic PCR using a primer set (arrows in panel (c)) to discriminate between the wild type and dsxF allele in homozygous (dsxF^{-/-}) heterozygous (dsxF^{+/-}) and wild type (wt) individuals.

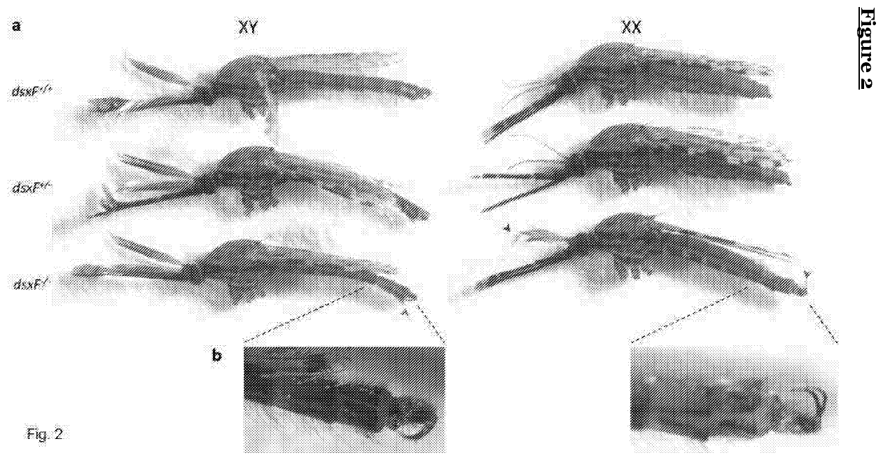

FIG. 2 shows morphological analysis of homozygous dsxF^{-/-} mutants. (a) Morphological appearance of genetic males and females heterozygous (dsxF^{+/-}) or homozygous (dsxF^{-/-}) for the exon 5 null allele. This assay was performed in a strain containing dominant RFP marker linked to the Y chromosome, whose presence permits unambiguous determination of male or female genotype. Anomalies in sexual morphology were observed only in dsxF^{-/-} genetic female mosquitoes. This group of XX individuals showed male-specific traits including a plumose antenna and claspers (arrows). This group also showed anomalies in the proboscis and accordingly they could not bite and feed on blood. Representative samples of each genotype are shown. (b) Magnification of the external genitalia. All dsxF^{-/-} females carried claspers, a male-specific characteristic. The claspers were dorsally rotated rather than in the normal ventral position.

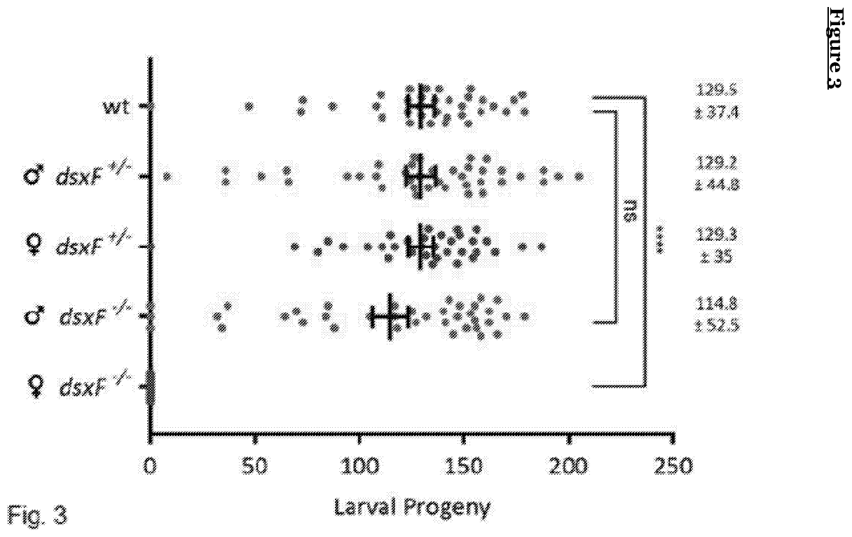

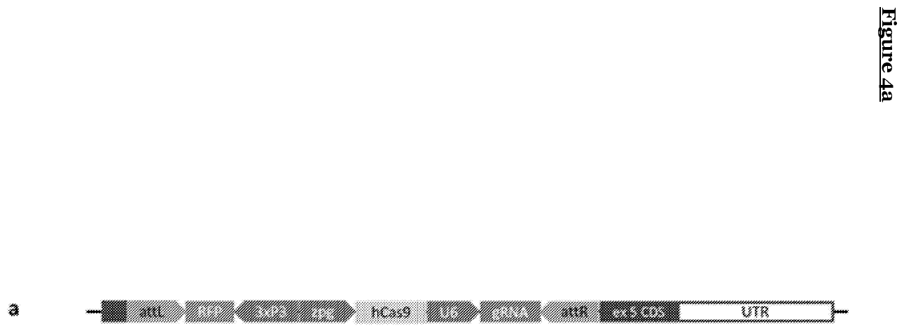

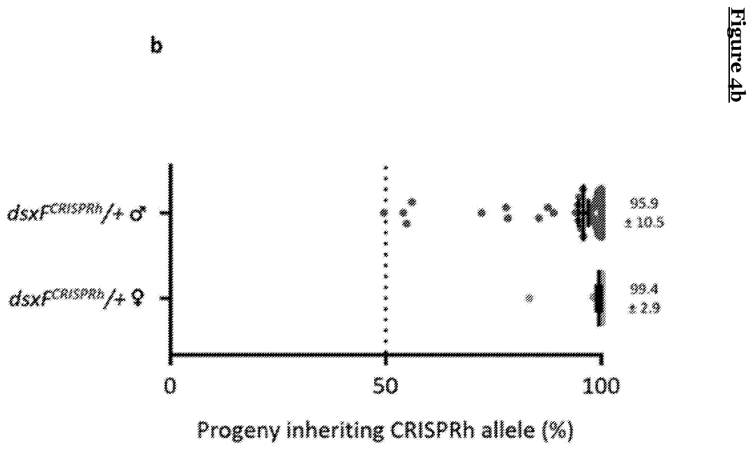

FIG. 3 shows the reproductive phenotype of dsxF mutants. Males and females dsxF^{-/-} and dsxF^{+/-} individuals were mated with the corresponding wild type sexes. Females were given access to a blood meal and subsequently allowed to lay individually. Fecundity was investigated by counting the number of larval progeny per lay (n≥43). Using wild type (wt) as a comparator the inventors saw no significant differences (‘ns’) in any genotype other than dsxF^{-/-} females, which were unable to feed on blood and therefore failed to produce a single egg (****, p<0.0001; Kruskal-Wallis test). Vertical bars indicate the mean and the s.e.m.

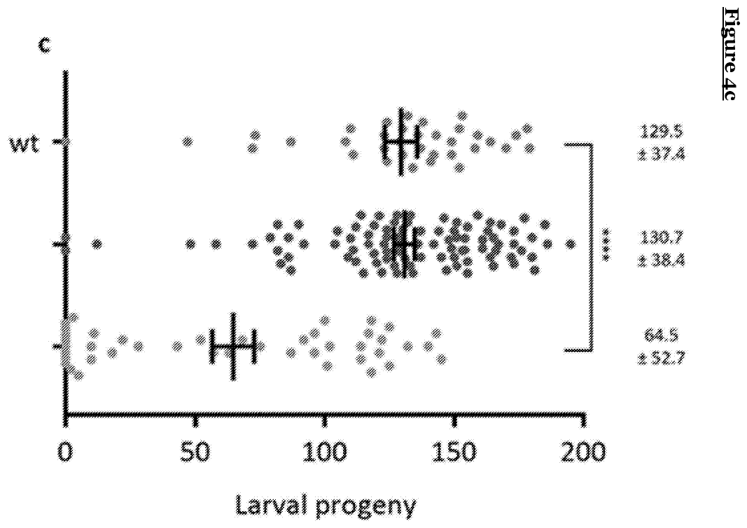

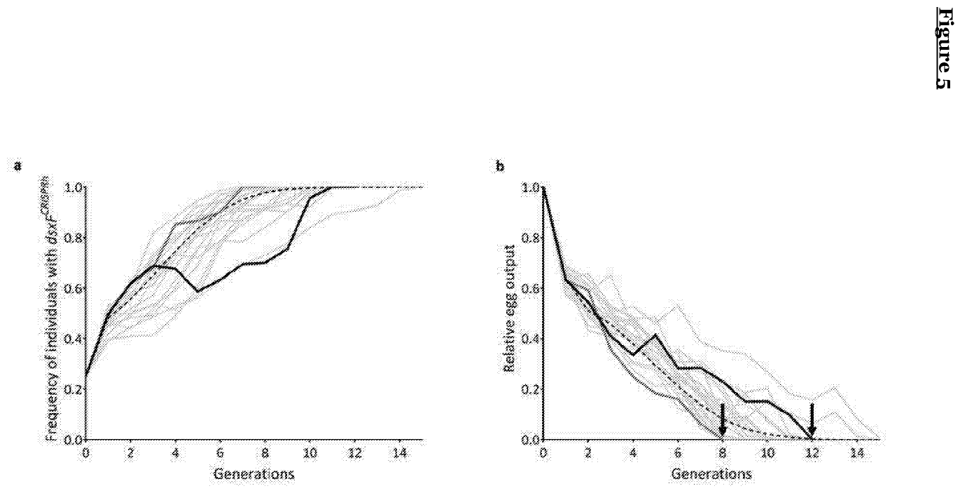

FIG. 4 shows the transmission rate of the dsxF^{CRISPRh} driving allele and fecundity analysis of heterozygous male and female mosquitoes. Male and female mosquitoes heterozygous for the dsxF^{CRISPRh} allele (a) (dsxF^{CRISPRh}/+) were analysed in crosses with wild type mosquitoes to assess the inheritance bias of the dsxF^{CRISPRh} drive construct (b) and for the effect of the construct on their reproductive phenotype (c). (b) Scattered plot of the transgenic rate observed in the progeny of dsxF^{CRISPRh}/+ female or male mosquitoes (n≥42) crossed to wild type individuals. Each dot represents the progeny derived from single females. Both male and female dsxF^{CRISPRh}/+ showed a high transmission rate of up to 100% of the dsxF^{CRISPRh} allele to the progeny. The transmission rate was determined by visual scoring among offspring of the RFP marker that is linked to the dsxF^{CRISPRh} allele. The dotted line indicates the expected Mendelian inheritance. Mean transmission rate(±s.e.m.) is shown (c) Scattered plot showing the number of larvae produced by single females from crosses of dsxF^{CRISPRh}/+ mosquitoes with wild type individuals after one blood meal. Mean progeny count(±s.e.m.) is shown. (****, p<0.0001; Kruskal-Wallis test).

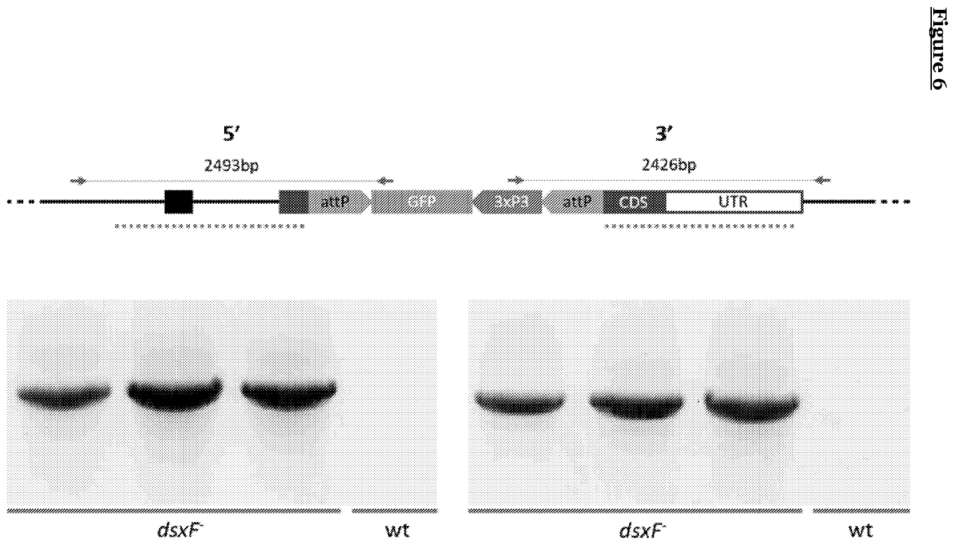

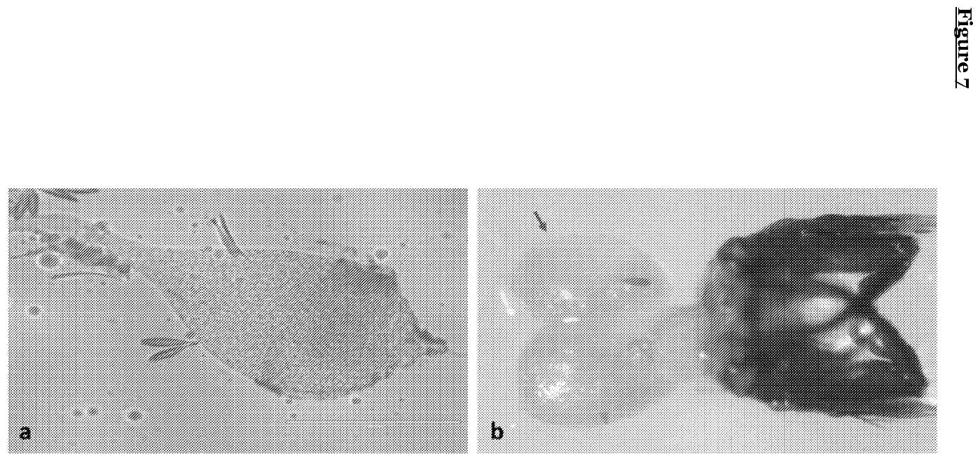

FIG. 5 shows the dynamics of the spread of the dsxF^{CRISPRh} allele and effect on population reproductive capacity. Two cages were set up with a starting population of 300 wild type females, 150 wild type males and 150 dsxF^{CRISPRh}/+ males, seeding each cage with a dsxF^{CRISPRh} allele frequency of 12.5%. The frequency of the dsxF^{CRISPRh} mosquitoes was scored for each generation (a). The drive allele reached 100% prevalence in both cage 2 (grey) and cage 1 (black) at generation 7 and 11 in agreement with a deterministic model (dotted line) that takes into account the parameter values retrieved from the fecundity assays. 20 stochastic simulations were run (faded grey lines) assuming a max population size of 650 individuals. (b) Total egg output deriving from each generation of the cage was measured and normalised relative to the output from the starting generation. Suppression of the reproductive output of each cage led the population to collapse completely (black arrows) by generation 8 (cage 2) or generation 12 (cage 1). Parameter estimates included in the model are provided in Table 1.

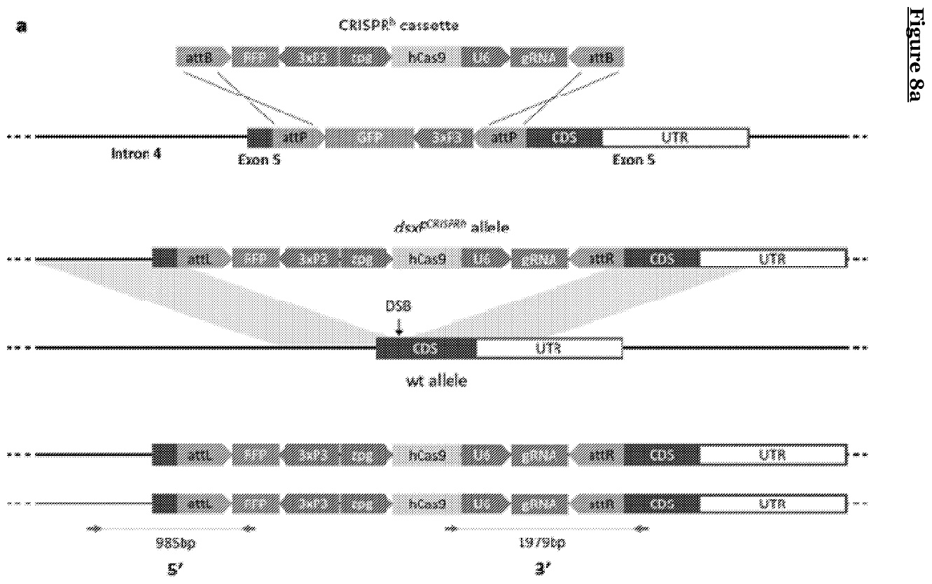

FIG. 6 shows the molecular confirmation of the correct integration of the HDR-mediated event to generate dsxF-. PCRs were performed to verify the location of the dsx φC31 knock-in integration. Primers (arrows) were designed to bind internal of the φC31 construct and outside of the regions used for homology directed repair (HDR) (dotted grey lines) which were included in the Donor plasmid K101. Amplicons of the expected sizes should only be produced in the event of a correct HDR integration. The gel shows PCRs performed on the 5′ (left) and 3′ (right) of 3 individuals for the dsx φC31 knock-in line (dsxF⁻) and wild type (wt) as a negative control.

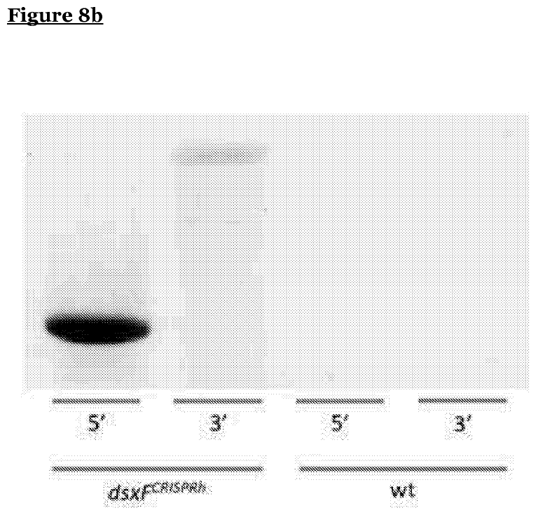

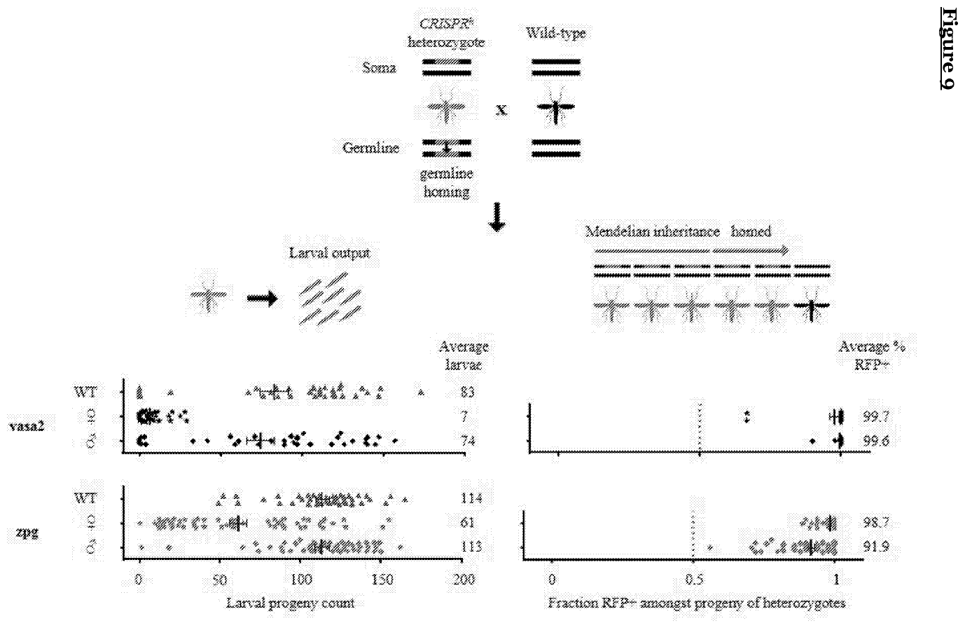

FIG. 7 shows the morphology of the dsxF^{-/-} internal reproductive organs. (a) Testis-like gonad from 3-days old female dsxF^{-/-} individual. There was no layer division between the cells and there was no evidence of sperm. (b) Dissections performed on dsxF^{-/-} genetic females revealed the presence of organs resembling accessory glands (arrow), a typical male internal reproductive organ.

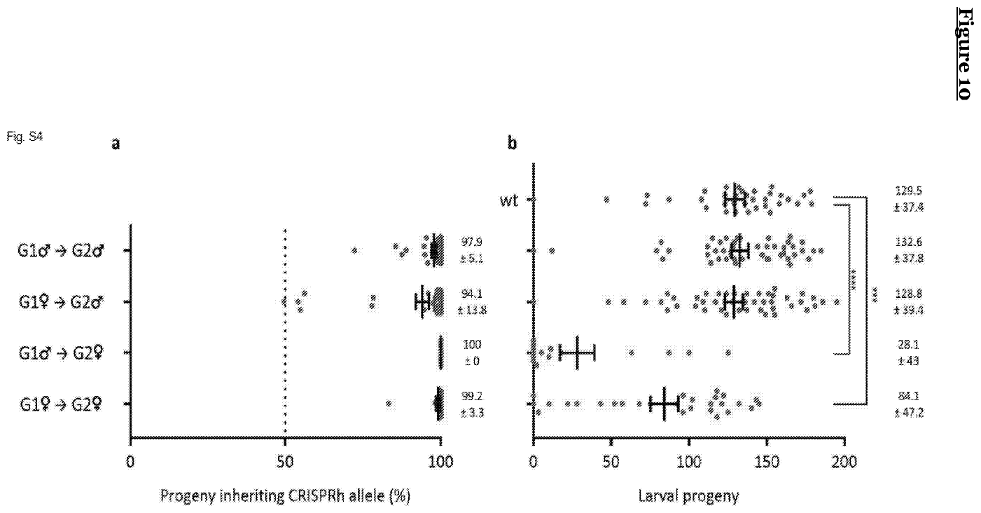

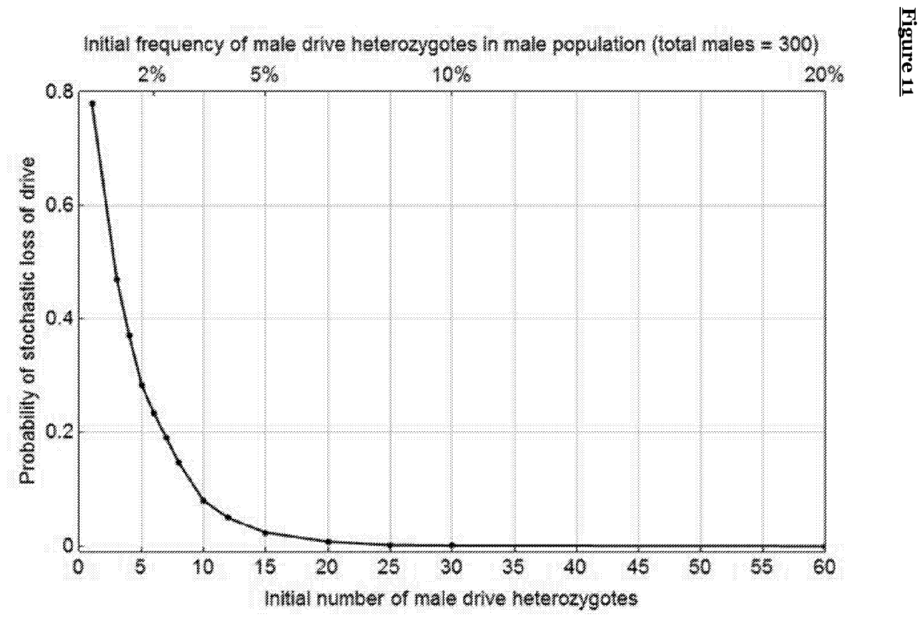

FIG. 8 shows the development of dsxF^{CRISPRh} drive construct and its predicted homing process and molecular confirmation of the locus. (a) The drive construct (CRISPR^{h} cassette) contained the transcription unit of a human codon-optimised Cas9 controlled by the germline-restrictive zpg promoter, the RFP gene under the control of the neuronal 3×P3 promoter and the gRNA under the control of the constitutive U6 promoter, all enclosed within two attB sequences. The cassette was inserted at the target locus using recombinase-mediated cassette exchange (RMCE) by injecting embryos with a plasmid containing the cassette and a plasmid containing a φC31 recombination transcription unit. During meiosis the Cas9/gRNA complex cleaves the wild type allele at the target locus (DSB) and the construct is copied across to the wild type allele via HDR (homing) disrupting exon 5 in the process. (b) Representative example of molecular confirmation of successful RMCE events. Primers (arrows) that bind components of the CRISPR^{h} cassette were combined with primers that bind the genomic region surrounding the construct. PCRs were performed on both sides of the CRISPR^{h} cassette (5′ and 3′) on many individuals as well as wild type controls (wt).

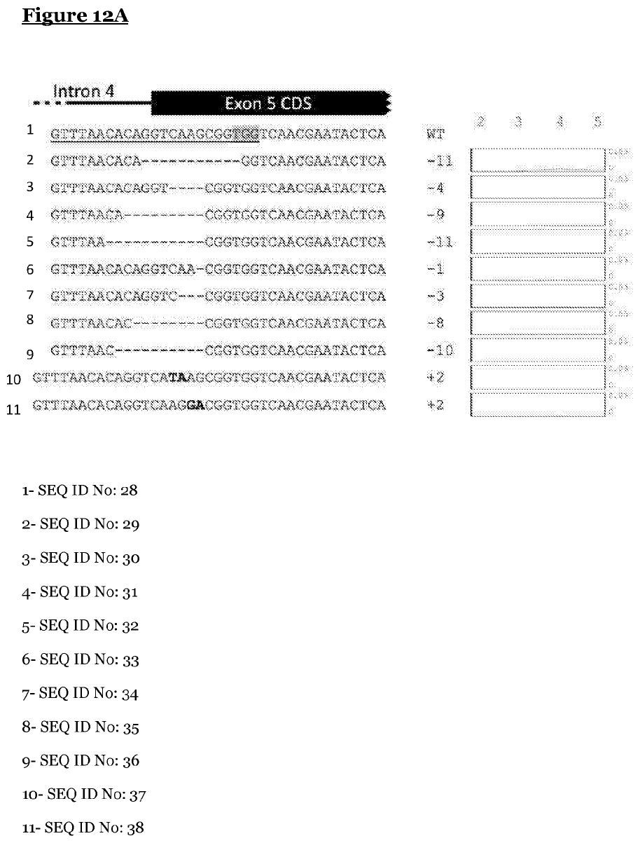

FIG. 9 shows the gene drives which were designed to express Cas9 under regulation of the promoter and terminator regions of zpg which show high rates of biased transmission and substantially improved fertility compared with the vasa2 promoter. Phenotypic assays were performed to measure fertility and transmission rates for each gene drive based upon the vasa and zpg promoters. The larval output was determined for individual drive heterozygotes crossed to wild type (left), and their progeny scored for the presence of DsRed linked to the construct (right). The average progeny count and transmission rate is also shown (±s.e.m.).

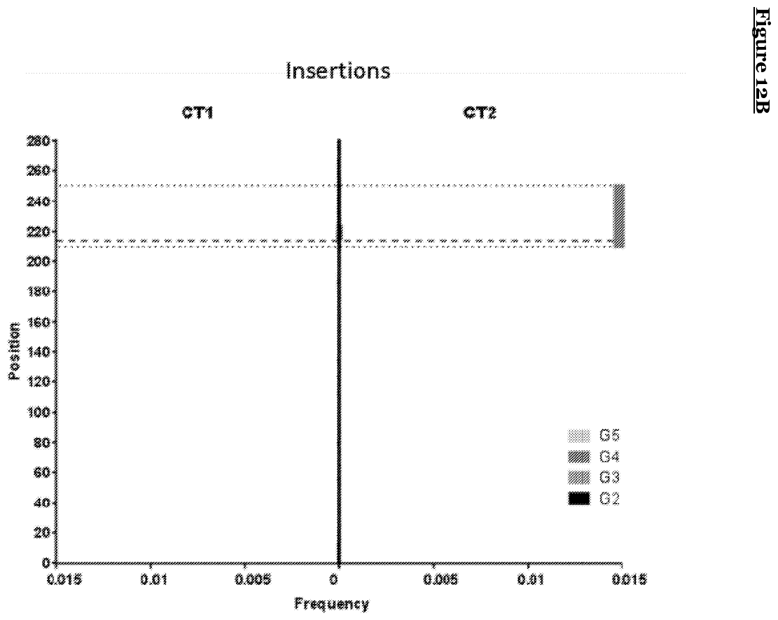

FIG. 10 shows the maternal or paternal inheritance of the dsxF^{CRISPRh} driving allele affect fecundity and transmission bias in heterozygotes. Male and female dsxF^{CRISPRh} heterozygotes (dsxF^{CRISPRh}/+) that had inherited a maternal or paternal copy of the driving allele were crossed to wild type and assessed for inheritance bias of the construct (a) and reproductive phenotype (b). (a) Progeny from single crosses (n≥15) were screened for the fraction that inherited DsRed marker gene linked to the dsxF^{CRISPRh} driving allele (e.g. G₁♂→G₂♀ represents a heterozygous female that received the drive allele from her father). Levels of homing were similarly high in males and females whether the allele had been inherited maternally or paternally. The dotted line indicates the expected Mendelian inheritance. Mean transmission rate(±s.e.m.) is shown. (b) Counts of hatched larvae for the individual crosses revealed a fertility cost in female dsxF^{CRISPRh} heterozygotes that was stronger when the allele was inherited paternally. Mean progeny count(±s.e.m.) is shown. (***, p<0.001; ****, p<0.0001; Kruskal-Wallis test).

FIG. 11 shows the probability of stochastic loss of the drive as a function of initial number of male drive heterozygotes. To calculate the probability of stochastic loss of the drive in the cage experiment setup, for each initial number (ho) of male drive heterozygous individuals, out of 1000 simulations of the stochastic cage model, the inventors recorded the number of times the drive was not present at 40 generations (and consequently population elimination did not occur). Each data point represents 1000 individual simulations of the stochastic cage model.

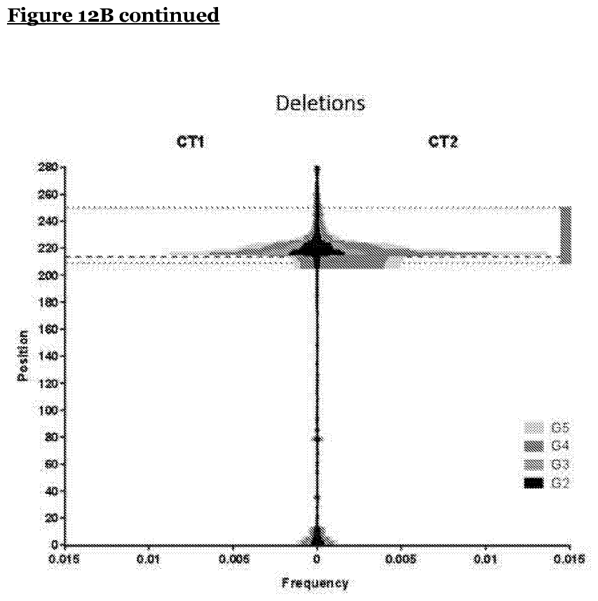

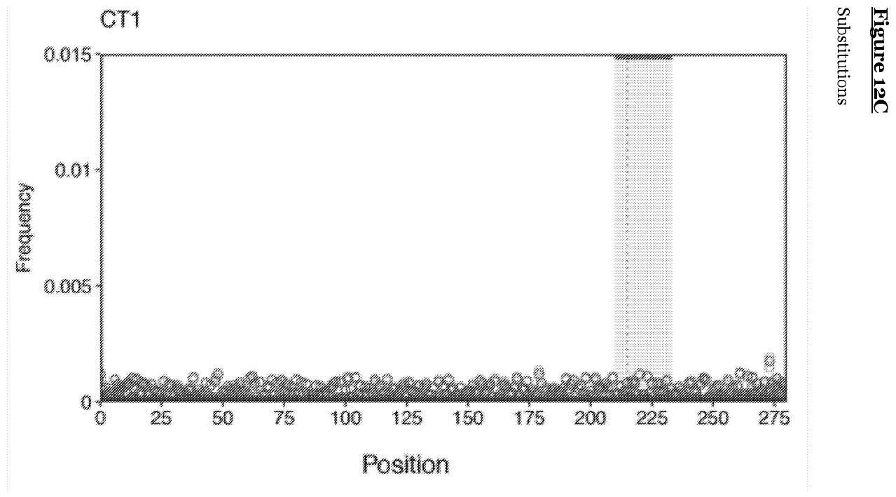

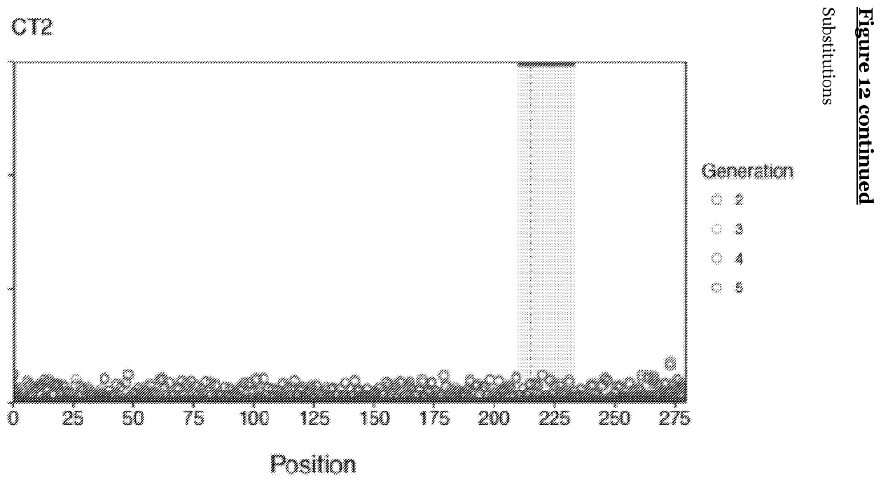

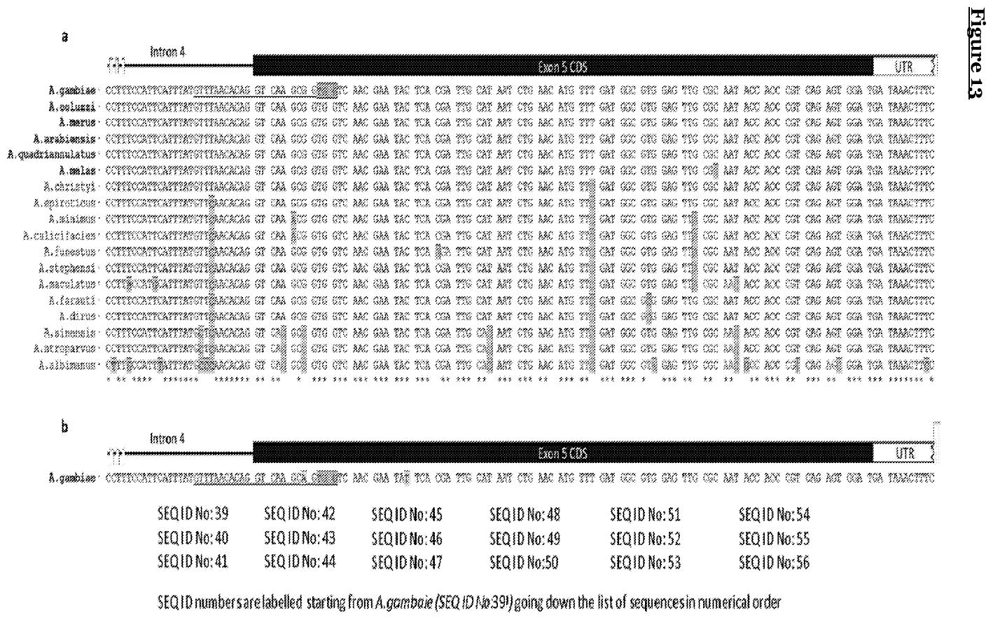

FIG. 12A-C show resistance plots variants and deletions in sequence. Pooled amplicon sequencing of the target site from 4 generations of the cage experiment (generations 2, 3, 4 and 5) revealed a range of very low frequency indels at the target site (a), none of which showed any sign of positive selection. Insertion, deletion and substitution frequencies per nucleotide position were calculated, as a fraction of all non-drive alleles, from the deep sequencing analysis for both cages. Distribution of insertions and deletions (b) in the amplicon is shown for each cage. Contribution of insertions and deletions arising from different generations is displayed. Significant change (p<0.01) in the overall indel frequency was observed in the region around the cut-site (dotted area±20 bp) for both cages. No significant changes were observed in the substitution frequency (c) around the cut-site (shaded area±20 bp) when compared with the rest of the amplicon, confirming that the gene drive did not generate any substitution activity at the target locus and that the laboratory colony is devoid of any standing variation in the form of SNPs within the entire amplicon.

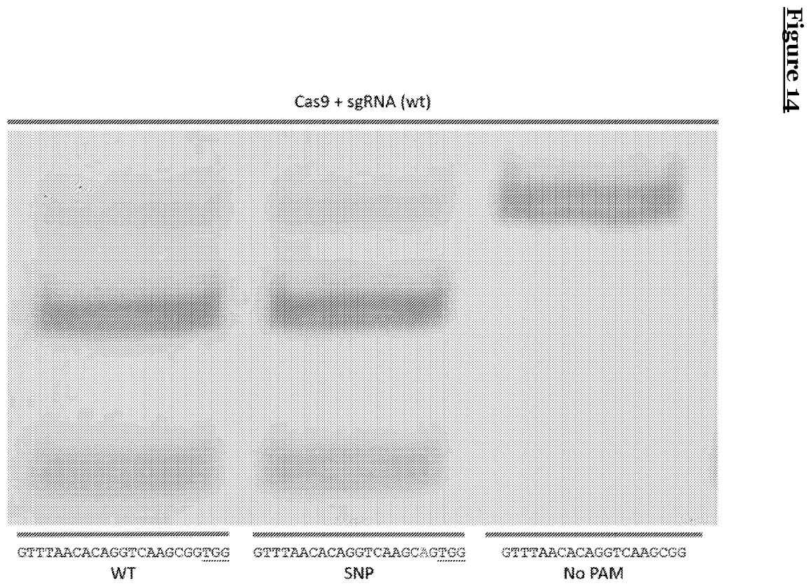

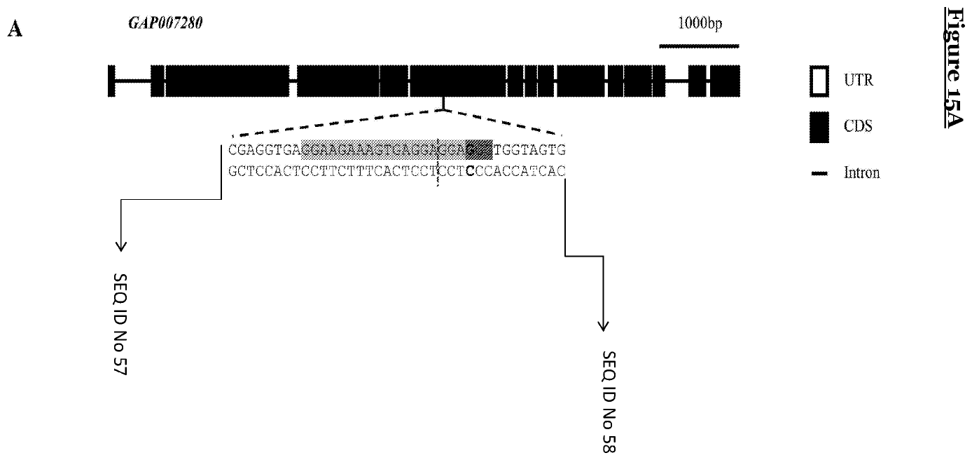

FIG. 13 shows a sequence comparison of the dsx female-specific exon 5 across members of the Anopheles genus and SNP data obtained from Anopheles gambiae mosquitoes in Africa. (a) Sequence comparison of the dsx intron 4-exon 5 boundary and the dsx female-specific exon 5 within the 16 Anopheline species¹⁶. The sequence of the intron 4-exon 5 boundary is completely conserved within the six species that form the Anopheles gambiae species complex (noted in bold). The gRNA used to target the gene is underlined and the PAM is highlighted in grey. (b) SNP frequencies obtained from 765 Anopheles gambiae mosquitoes captured across Africa¹⁷. Across the dsx female-specific Exon 5 there are only 2 SNP variants (noted with arrows) with frequencies of 2.9% (the SNP in the gRNA-complementary sequence) and 0.07%—SEQ ID No: 59.

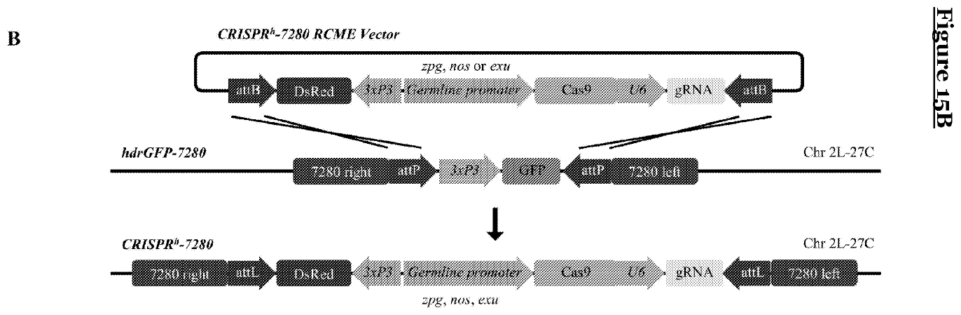

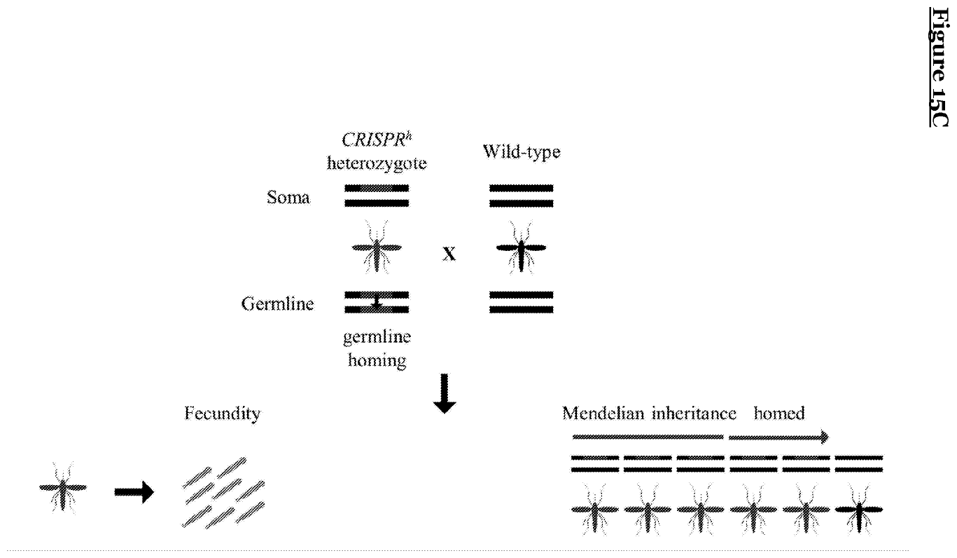

FIG. 14 shows an in vitro cleavage assay testing the efficiency of the gRNA in the dsxF^{CRISPRh} gene drive to cleave the dsx exon 5 target site with the SNP found in wild populations in Africa. An in vitro cleavage assay using an RNP complex of Cas9 enzyme and the gRNA used in this study was performed against linearised plasmids containing either wild type (WT) target site in dsx exon 5 (SEQ ID No: 60) or the same site containing the single SNP found in wild caught populations (SNP) (SEQ ID No: 61). Products of the in vitro cleavage assay were purified and analysed on a gel. Both the WT and SNP-containing target sites are susceptible to the cleavage activity of the RNP complex as shown by the diminished high molecular band and the presence of the two cleavage products of the expected size. A dsx exon 5 target site containing the WT sequence complementary to the gRNA but without the PAM sequence was used as a control (‘no PAM’) (SEQ ID No: 62).

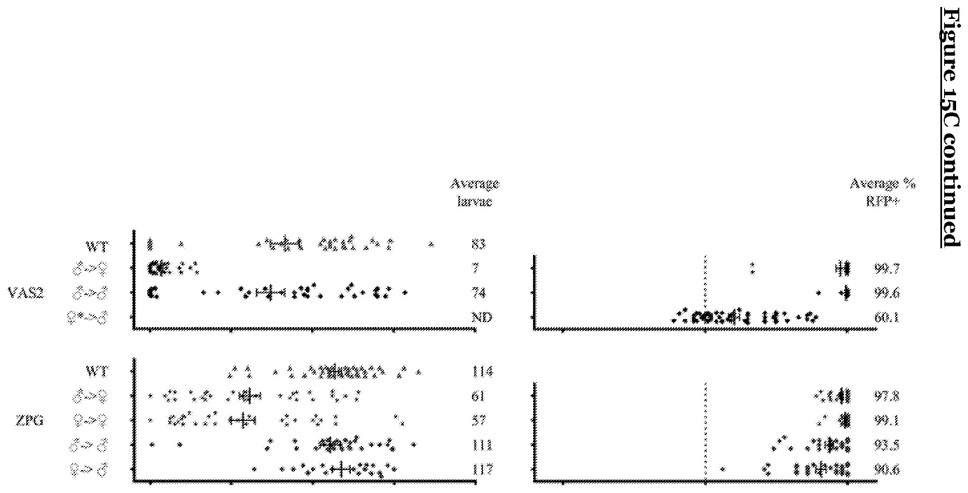

FIG. 15A-D. Gene drives designed to express Cas9 under regulation of zpg, nos and exu germline promoters show high rates of biased transmission and substantially improved fertility compared with the vas2 promoter. (a) The haplosufficient female fertility gene, AGAP007280, and its target site in exon 6 (highlighted in grey), showing the protospacer-adjacent motif (highlighted in teal) and cleavage site (red dashed line). (b) CRISPR^{h} alleles were inserted at the target in AGAP007280 using φC31-recombinase mediated cassette exchange (RCME). Each CRISPR^{h} RCME vector was designed to contain Cas9 under transcriptional control of the nos, zpg or exu germline promoter, a gRNA targeted to AGAP007280 under the control of the ubiquitous U6 PolIII promoter, and a 3×P3::DsRed marker. (c) Phenotypic assays were performed to measure fertility and transmission rates for each of three drives. The larval output was determined for individual drive heterozygotes crossed to wild-type (left), and their progeny scored for the presence of DsRed linked to the construct (right). Males and females were further separated by whether they had inherited the CRISPR^{h} construct from either a male or female parent. For example, ♂→♀ denotes progeny and transmission rates of a heterozygous CRISPR^{h} female that had inherited the drive allele from a heterozygous male. The average progeny count and transmission rate is also shown (±s.e.m.). High levels of homing were observed in the germline of zpg-CRISPR^{h} and nos-CRISPR^{h} males and females, however the exu promoter generated only moderate levels of homing in the germline of males but not females. Counts of hatched larvae for the individual crosses revealed improvements in the fertility of heterozygous females containing CRISPR^{h} alleles based upon zpg, nos and exu promoters compared to the vas2 promoter. In each case, the average number of hatched larvae improved relative to wild-type controls, or equivalent CRISPR^{h} heterozygous males (whereby no fertility cost is anticipated). Phenotypic assays were performed on G2 and G3 for zpg, G3 and G4 for nos, and ˜G15 for exu. ♀* denotes vas2-CRISPR^{h} females that were heterozygous with a resistance (R1) allele, these were used because heterozygous vas2-CRISPR^{h} females are usually sterile.

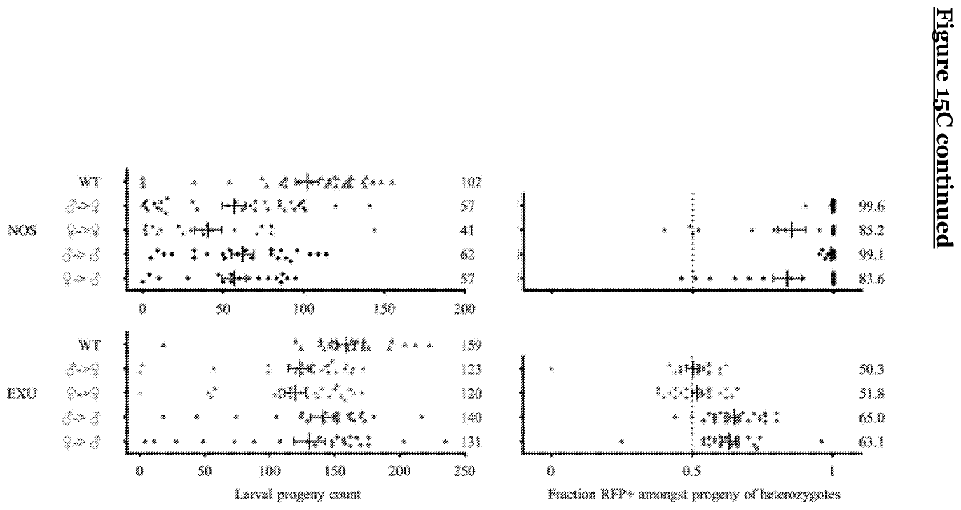

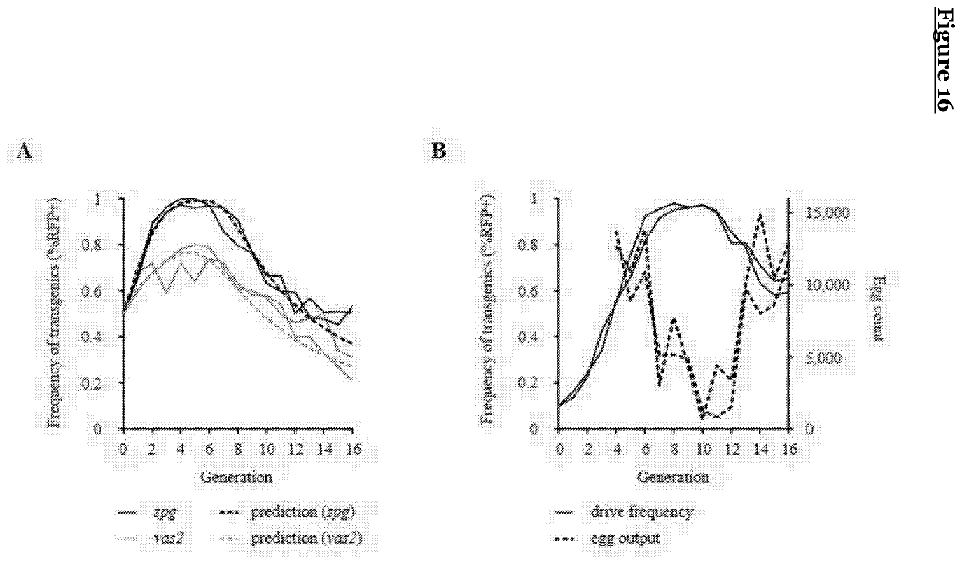

FIG. 16A-B shows CRISPRh gene drives based upon the zpg promoter spread throughout entire caged populations of the malaria mosquito and cause a substantial reduction in reproductive output. (a) Equal numbers of CRISPRh/+ and WT individuals were used to initiate replicate caged populations, and the frequency of drive-modified mosquitoes was recorded each generation by screening larval progeny for the presence of DsRed linked to the CRISPRh construct. Solid lines show results from two replicate cages for zpg (black) and previous results for vas2 (grey). Deterministic predictions are shown for zpg (black dashed line) and vas2 (grey dashed line) based on observed parameter values for homing in males (zpg=83.6%, vas2=98.4%), homing in females (zpg=93.4%, vas2=98.4%), heterozygous female fitness (zpg=83%, vas2=9.3%), homozygous females completely sterile, and assuming no fitness cost in males. (b) A lower release rate of 10% CRISPRh/+ was used to initiate two further replicate populations in which the frequency of drive-modified mosquitoes (solid line) and counts of the entire egg progeny (dashed line) were recorded each generation.

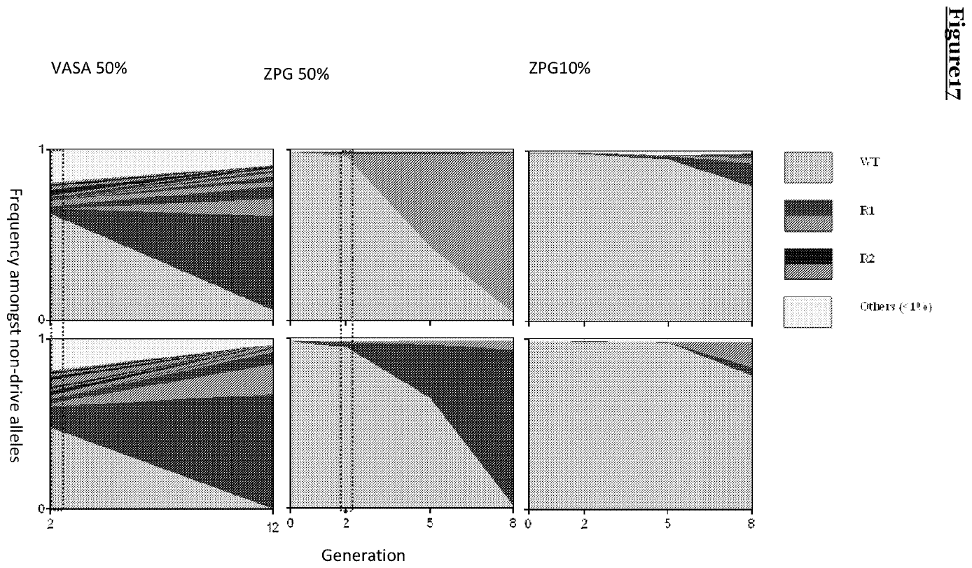

FIG. 17 shows a change in frequency of wild-type, resistant and non-resistant alleles during spread of vas2- and zpg-based gene drives in caged releases. The nature and frequency of wild-type and mutant alleles was determined for several early and late generations by amplicon sequencing across the target site in pooled samples of entire caged populations. Alleles above 1% frequency in any generation are identified as wild-type (grey), R1 (alternating red and pink) and R2 (alternating blue and violet), the remaining alleles that are individually below 1% frequency across generations are grouped together (yellow). The left-most column shows previously published data for allele frequencies in replicate cages of vas2-based drives released at 50% frequency (Hammond & Kyrou et al. 2017), the middle and right-most columns show new allele frequency data for replicate cages of zpg-based drives released at 50% and 10%, respectively. Already at generation 2 of the 50% releases (inside dotted boxes), 14 different mutant alleles were present at more than 1% frequency in the vas2 cages compared to just two alleles in each of the zpg cages. All R1 alleles highlighted in zpg cages were previously confirmed to restore fertility, whereas R1 alleles highlighted in vas2 cages include all in-frame mutations whether or not they have been confirmed to restore fertility.

### EXAMPLES

The invention described herein relies on inserting site-specific nuclease genes into a locus of choice, in formations that both confer some trait of interest on an individual and lead to a biased inheritance of the trait. The approach relies on “homing” leading to suppression. The invention is focused on population suppression, whereby the gene drive construct is designed to insert within a target gene in such a way that the gene product, or a specific isoform thereof, is disrupted. To build the nuclease-based gene drive of the invention, the nuclease gene is inserted within its own recognition sequence in the genome such that a chromosome containing the nuclease gene cannot be cut, but chromosomes lacking it are cut. When an individual contains both a nuclease-carrying chromosome and an unmodified chromosome (i.e. heterozygous for the gene drive), the unmodified chromosome is cut by the nuclease. The broken chromosome is usually repaired using the nuclease-containing chromosome as a template and, by the process of homologous recombination, the nuclease is copied into the targeted chromosome. If this process, called “homing”, is allowed to proceed in the germline, then it results in a biased inheritance of the nuclease gene, and its associated disruption, because sperm or eggs produced in the germline can inherit the gene from either the original nuclease-carrying chromosome, or the newly modified chromosome.

Due to the negative reproductive load the gene drive imposes, selection can be expected to occur for resistant alleles. The most likely source of such resistance is sequence variation at the target site that prevents the nuclease cutting yet at the same time permits a functional product from the target gene. Such variation can pre-exist in a population or can be created by activity of the nuclease itself—a small proportion of cut chromosomes, rather than using the homologous chromosome as a template, can instead be repaired by end-joining (EJ), which can introduce small insertions or deletions (“indels”) or base substitutions during the repair of the target site. In-frame indels or conservative substitutions might be expected to show selection in the presence of a gene drive. The inventors have previously observed target site resistance in cage experiments (data not shown) and found that end-joining in chromosomes of the early embryo, due to parentally-deposited nuclease, was likely to be the predominant source of the resistant alleles at the target site.

In mitigating and preventing the emergence of resistant alleles, the strategy being investigated by the inventors involves reducing the embryonic source of end-joining mutations by expressing the nuclease from promoters that show tighter, germline-restricted expression and less maternal and paternal deposition, e.g. nanos (nos), zero population (zpg), and exuperentia (exu).

Materials and Methods

Pooled Amplicon Sequencing of Caged Experiments

Pooled amplicon sequencing was performed as described before in Hammond and Kyrou (2017)⁶. Up to 600 adults were homogenized from the cage trial experiments at generations 0, 2, 5, and 8, and extracted in pooled groups using the Wizard Genomic DNA purification kit (Promega). A 332 bp locus spanning the target site was amplified from 90 ng of each genomic sample using KAPA HiFi HotStart Ready Mix PCR kit (Kapa Biosystems) in 50 ul reactions. Primers were designed to include the Illumina Nextera Transposase Adapters (underlined),

7280-Illumina-F
(TCGTCGGCAGCGTCAGATGTGTATAAGAGACAGGGAGAAGGTAAATGC
GCCAC-SEQ ID No: 63)
and
7280-Illumina-R
(GTCTCGTGGGCTCGGAGATGTGTATAAGAGACAGGCGCTTCTACACTC
GCTTCT-SEQ ID No: 64)

for downstream library preparation and sequencing. The primers were annealed at 68° C. for 20 seconds to minimize off target amplification. In order to maintain an accurate representation of the allele frequencies at the target site, 25 μL of the PCR reaction was removed at 20 cycles, whilst the reaction was non-saturated, and stored at −20° C. The remnant 25 μL was run for an additional 20 cycles to verify the reaction on an agarose gel. The non-saturated samples were purified with AMPure XP beads (Beckman Coulter) and used in a second PCR reaction in which dual indices and Illumina sequencing adapters from the Nextera XT Index Kit were added according to the Illumina 16S Metagenomic Sequencing Library Preparation protocol (Part #15044223). The PCR was purified again with AMPure XP beads and validated with Agilent Bioanalyzer 2100. The normalized libraries were sequenced in a pooled reaction at a concentration of 10 pM on an Illumina Nano flowcell v2 using the Illumina MiSeq instrument with a 2×250 bp paired-end run.

Use of zpg Promoter to Drive Cas9 Expression in Gene Drive Constructs

The gene drive construct targeting dsxF is identical in design to that described in Hammond et al. except for the promoter and 3′ UTR surrounding the Cas9 gene—where previously these were from the ortholog of vasa (AGAP008578), in the current construct these are replaced by 1074 bp upstream and 1034 bp downstream of the germline-specific gene AGAP006241, the putative ortholog of zero population growth (zpg). The inventors performed a comparison of the fertility and homing rates in individuals heterozygous vasa- and zpg-driven gene CRISPR^{h} constructs at the exact same target locus in AGAP007280, previously described in Hammond et al. (FIG. 9). Counts of hatched larvae for the individual crosses revealed improvements in the fertility of heterozygous females containing CRISPR^{h} alleles based upon zpg, where larval output was 50-53% of wild type control compared to just 8.4% for vasa. No fertility effect was observed in males. To assess the level of homing, drive heterozygotes were crossed to wild type, allowed to lay individually, and their progeny scored for the presence of DsRed linked to the construct. Transmission rates for the zpg constructs exceeded 91.9% in males and 98.7% in females—the previously observed rates for vasa constructs were 99.6% in males and 97.7% in females.

Probability of Stochastic Loss of the Drive as a Function of Initial Number of Male Drive Heterozygotes

To calculate the probability of stochastic loss of the drive in the cage experiment setup, for each initial number (ho) of male drive heterozygous individuals, out of 1000 simulations of the stochastic cage model, The inventors recorded the number of times the drive was not present at 40 generations (and consequently population elimination did not occur). Each data point represents woo individual simulations of the stochastic cage model (FIG. 11).

In Vitro Cleavage Assay Against Wild Type and SNP Variant Target Site

The inventors performed an in vitro cleavage assay to test the ability of the gRNA used in this study to cleave the target site that incorporates the SNP found in wild populations in Africa (FIG. 14). Using Golden Gate cloning and primers modified to carry suitable overhangs, the inventors introduced the two target sequences separately into a 2 kb plasmid. As a control, the inventors also prepared a plasmid that carries a modified version of the dsx target site without the SNP that lacks the PAM sequence, necessary for Cas9 cleavage. All three vectors were linearized and verified on a gel prior to the cleavage assay. For the cleavage assay the inventors used a ready-to-use sgRNA provided by Synthego (USA) and S. pyogenes Cas9 nuclease in the form of enzyme (NEB). To form ribonucleoprotein particles (RNPs) the inventors mixed same molar ratios of the sgRNA and the Cas9 protein into a 40 μl reaction to a final concentration of 400 nM and left to incubate at room temperature for 10 minutes. The linearized substrate was added to the reactions in a final concentration of 40 nM, in a final volume of 50 μl and left to incubate at 37° C. for 30 minutes. Proteinase K was added to stop the reaction and 20 μl were verified on a gel.

Amplification of Promoter and Terminator Sequences

The published Anopheles gambiae genome sequence provided in Vectorbase (Giraldo-Calderon et al, 2015) was used as a reference to design primers in order to amplify the promoters and terminators of the three Anopheles gambiae genes: AGAP006098 (nanos), AGAP006241 (zero population growth) and AGAP007365 (exuperantia).

Using the primers provided in Table 3 the inventors performed PCRs on 40 ng of genomic material extracted from wild type mosquitoes of the G3 strain using the Wizard Genomic DNA purification kit (Promega). The primers were modified to contain suitable Gibson assembly overhangs (underlined) for subsequent vector assembly. Promoter and terminator fragments were 2092 bp and 601 bp for nos, 1074 bp and 1034 bp for zpg, and 849 and 1173 bp for exu, respectively. The sequences of all regulatory fragments can be found in Table 4.

Generation of CRISPR^{h} Drive Constructs

The inventors modified available template plasmids used previously in Hammond et al. (2016)² to replace and test alternative promoters and terminators for expressing the Cas9 protein in the germline of the mosquito. p16501, which was used in that study carried a human optimised Cas9 (hCas9) under the control of the vas22 promoter and terminator, an RFP cassette under the control of the neuronal 3×P3 promoter and a U6:sgRNA cassette targeting the AGAP007280 gene in Anopheles gambiae.

The hCas9 fragment and backbone (sequence containing 3×P3::RFP and a U6::gRNA cassette), were excised from plasmid p16501 using the restriction enzymes XhoI+PacI and AscI+AgeI respectively. Gel electrophoresis fragments were then re-assembled with PCR amplified promoter and terminator sequences of zpg, nos or exu by Gibson assembly to create new CRISPR^{h} vectors named p17301 (nos), p17401 (zpg) and p17501 (exu).

Transformation of Drive Constructs into Genome at AGAP007280

CRISPR^{h} constructs containing Cas9 under control of the zpg, nos and exu promoters were inserted into an hdrGFP docking site previously generated at the target site in AGAP007280 (Hammond et al. 2016).

Anopheles gambiae mosquitoes of the hdrGFP-7280 strain were reared under standard conditions of 80% relative humidity and 28° C., and freshly laid embryos used for microinjections as described before (Fuchs et al, 2013). Freshly-laid embryos were microinjected as described before (Fuchs et al, 2013). Recombinase-mediated cassette exchange (RCME) reactions were performed by injecting each of the new CRISPR^{h} constructs into embryos of the hdrGFP docking line that was previously generated at the target site in AGAP007280 (Hammond et al. 2016). For each construct, embryos were injected with solution containing CRISPR^{h} (400 ng/μl) and a vas2::integrase helper plasmid (400 ng/μl) (Volohonsky et al, 2015). Surviving Gₒ larvae were crossed to wild type transformants identified by a change from GFP (present in the hdrGFP docking site) to DsRed linked the CRISPR^{h} construct that should indicate successful RCME.

Molecular Confirmation of Gene Targeting and Cassette Integration

Successful RMCE integration of CRISPR^{h} constructs into the genome at AGAP007280 were confirmed by PCR using genomic DNA extracted using the Wizard Genomic DNA purification kit (Promega). Primers binding the integrated cassette (hCas9-F7 and RFP2qF) were used with primers that bind the neighbouring genomic integration site in AGAP007280 (Seq-7280-F and Seq-7280-R) to verify the presence but also the orientation of the CRISPR^{h} cassette. Primer sequences can be found in (Supplementary Table S2).

Caged Experiments

The cage trials were performed following the same principle described before in Hammond et al. (2016). Briefly, heterozygous zpg-CRISPR^{h} that had inherited the drive from a female parent were mixed with age-matched wild type at L1 at 10% or 50% frequency of heterozygotes. At the pupal stage, 600 were selected to initiate replicate cages for each initial release frequency. Adult mosquitoes were left to mate for 5 days before they were blood fed on anesthetized mice. Two days after, the mosquitoes were left to lay in a 300 ml egg bowl filled with water and lined with filter paper. Each generation, all eggs were allowed two days to hatch and 600 randomly selected larvae were screened to determine the transgenic rate by presence of DsRed and then used to seed the next generation. From generation 4 onwards, adults were blood-fed a second time and the entire egg output photographed and counted using JMicroVision V1.27. Larvae were reared in 2 L trays in 500 ml of water, allowing a density of 200 larvae per tray. After recovering progeny, the entire adult population was collected and entire samples from generation 0, 2, 5, and 8 were used for pooled amplicon sequence analysis.

Phenotypic Assays to Measure Fertility and Rates of Homing

Heterozygous CRISPR^{h}/+ mosquitoes from each of the three new lines zpg-CRISPR^{h}, nos-CRISPR^{h}, zpg-CRISPR^{h}, were mated to an equal number of wild type mosquitoes for 5 days in reciprocal male and female crosses. Females were blood fed on anesthetized mice on the sixth day and after 3 days, a minimum of 40 were allowed to lay individually into a 25-ml cup filled with water and lined with filter paper. The entire larval progeny of each individual was counted and a minimum of 50 larvae were screened to determine the frequency of the DsRed that is linked to the CRISPR^{h} allele by using a Nikon inverted fluorescence microscope (Eclipse TE200). Females that failed to give progeny and had no evidence of sperm in their spermathecae were excluded from the analysis. Statistical differences between genotypes were assessed using the Kruskal-Wallis test.

Population Genetics Model

To model the results of the cage experiments, the inventors used discrete-generation recursion equations for the genotype frequencies, treating males and females separately. F_ij (t) and M_ij (t) denote the frequency of females (or males) of genotype i/j in the total female (or male) population. The inventors considered three alleles, W (wildtype), D (driver) and R (non-functional resistant), and therefore six genotypes.

Homing

Adults of genotype W/D produce gametes at meiosis in the ratio W:D:R as follows:

(1−d_{f})(1−u_{f}):d_{f}:(1−d_{f})u_{f} in females

(1−dₘ)1−uₘ):dₘ:(1−dₘ)uₘ in males

Here, d_f and d_m are the rates of transmission of the driver allele in the two sexes and u_f and u_m are the fractions of non-drive gametes that are non-functional resistant (R alleles) from meiotic end-joining. In all other genotypes, inheritance is Mendelian. Fitness. Let w_ij≤1 represent the fitness of genotype i/j relative to w_WW=1 for the wild-type homozygote. The inventors assume no fitness effects in males. Fitness effects in females are manifested as differences in the relative ability of genotypes to participate in mating and reproduction. The inventors assume the target gene is needed for female fertility, thus D/D, D/R and R/R females are sterile; there is no reduction in fitness in females with only one copy of the target gene (W/D, W/R).

Parental Effects

The inventors consider that further cleavage of the W allele and repair can occur in the embryo if nuclease is present, due to one or both contributing gametes derived from a parent with one or two driver alleles. The presence of parental nuclease is assumed to affect somatic cells and therefore female fitness but has no effect in germline cells that would alter gene transmission. Previously, embryonic EJ effects (maternal only) were modelled as acting immediately in the zygote [1, 2]. Here, the inventors consider that experimental measurements of female individuals of different genotypes and origins show a range of fitnesses, suggesting that individuals may be mosaics with intermediate phenotypes. The inventors therefore model genotypes W/X (X=W, D, R) with parental nuclease as individuals with an intermediate reduced fitness w_{WX}¹⁰, w_{WX}⁰¹, or w_{WX}¹¹ depending on whether nuclease was derived from a transgenic mother, father, or both. The inventors assume that parental effects are the same whether the parent(s) had one or two drive alleles. For simplicity, a baseline reduced fitness of w₁₀, w₀₁, w₁₁ is assigned to all genotypes W/X (X=W, D, R) with maternal, paternal and maternal/paternal effects, with fitness estimated as the product of mean egg production values and hatching rates relative to wild-type in Table 1 in the deterministic model. In the stochastic version of the model, egg production from female individuals with different parentage is sampled with replacement from experimental values.

TABLE 1
Parameters for stochastic cage model

| Parameter | Estimate | Method of estimation |
|---|---|---|
| Mating probability | 0.85 for heterozygotes; 0 for D/D, | Estimated from |
| | D/R and R/R homozygotes | Hammond et al. 2017 |
| Egg production from | Mean 137.4. Sampling with | From assays of mated |
| wildtype female | replacement of observed values | females |
| (no parental nuclease) | (10, 61, 96, 98, 111, 111, 113, 127, 128, | |
| | 129, 132, 132, 134, 135, 137, 138, 138, | |
| | 139, 142, 142, 146, 146, 149, 152, 152, | |
| | 152, 158, 160, 162, 164, 170, 179, 186, | |
| | 189, 191) | |
| Egg production from | Mean 118.96. Sampling with | From assays of mated |
| W/D heterozygote female | replacement of observed values (12, | females |
| (nuclease from ♀) | 31, 76, 90, 96, 100, 106, 106, 107, 113, | |
| | 117, 118, 119, 130, 133, 136, 136, 136, | |
| | 137, 138, 139, 142, 143, 145, 146, 148, | |
| | 157, 174) | |
| Egg production from | Mean 59.67. Sampling with | From assays of mated |
| W/D heterozygote female | replacement of observed values | females |
| (nuclease from ♂) | (0, 0, 0, 0, 0, 34, 47, 50, 65, 105, 113, | |
| | 115, 115, 125, 126) | |
| Hatching probability, | 0.941 | From assays of mated |
| wildtype female | | females |
| (no parental nuclease) | | |
| Hatching probability, | 0.707 | From assays of mated |
| W/D heterozygote female | | females |
| (nuclease from ♀) | | |
| Hatching probability, | 0.47 | From assays of mated |
| W/D heterozygote female | | females |
| (nuclease from ♂) | | |
| Probability of emergence | 0.8708 | Average of observations |
| from pupa (survival from | | over all generations and |
| larva) | | both cage experiments |
| Drive in | 0.9985 | Observed fraction |
| W/D females | | transgenic from assays |
| Drive in | 0.9635 | Observed fraction |
| W/D males | | transgenic from assays |
| Meiotic EJ parameter | 0.4685 | Estimated from |
| (fraction non-drive alleles | | Hammond et al. 2016 |
| that are resistant) | | |

Recursion Equations

The inventors firstly considered the gamete contributions from each genotype, including parental effects on fitness. In addition to W and R gametes that are derived from parents that have no drive allele and therefore have no deposited nuclease, gametes from W/D females and W/D, D/R and D/D males carry nuclease that is transmitted to the zygote, and these are denoted as W\hat{ }*, D\hat{ }*, R\hat{ }*. The proportion of type i alleles in eggs produced by females participating in reproduction are given in terms of male and female genotype frequencies below. Frequencies of mosaic individuals with parental effects (i.e., reduced fitness) due to nuclease from mothers, fathers or both are denoted by superscripts 10, 01 or 11.

e_{W}=(F_{WW}+w_{WW}¹⁰F_{WW}¹⁰+w_{WW}⁰¹F_{WW}⁰¹+w_{WW}¹¹F_{WW}¹¹+(F_{WR}+w_{WR}¹⁰F_{WR}¹⁰+w_{WR}⁰¹F_{WR}⁰¹+w_{WR}¹¹F_{WR}¹¹)/2)/w̅_{f}

e_{R}=1/2(F_{WR}+w_{WR}¹⁰F_{WR}¹⁰+w_{WR}⁰¹F_{WR}⁰¹+w_{WR}¹¹F_{WR}¹¹)/w̅_{f}

e*_{W}=(1−d_{f})(1−u_{f})(w_{WD}¹⁰F_{WD}¹⁰+w_{WD}⁰¹F_{WD}⁰¹+w_{WD}¹¹F_{WD}¹¹)/w̅_{f}

e*_{D}=d_{f}(w_{WD}¹⁰F_{WD}¹⁰+w_{WD}⁰¹F_{WD}⁰¹+w_{WD}¹¹F_{WD}¹¹)/w̅_{f}

e*_{R}=(1−d_{f})u_{f}(w_{WD}¹⁰F_{WD}¹⁰+w_{WD}⁰¹F_{WD}⁰¹+w_{WD}¹¹F_{WD}¹¹)/w̅_{f}

The proportions sᵢ of type i alleles in sperm are:

s_{W}=(M_{WW}+M_{WW}¹⁰+M_{WW}⁰¹+M_{WW}¹¹+(M_{WR}+M_{WR}¹⁰+M_{WR}⁰¹+M_{WR}¹¹)/2)/w̅ₘ

s_{R}=(M_{RR}+(M_{WR}+M_{WR}¹⁰+M_{WR}⁰¹+M_{WR}¹¹)/2)/w̅ₘ

s*_{W}=(1−dₘ)(1−uₘ)(M_{WD}¹⁰+M_{WD}⁰¹+M_{WD}¹¹)/w̅ₘ

s*_{D}=(M_{DD}+M_{DR}/2+dₘ(M_{WD}¹⁰+M_{WD}⁰¹+M_{WD}¹¹))/w̅ₘ

s*_{R}=(M_{DR}/2+(1−dₘ)uₘ(M_{WD}⁰¹+M_{WD}¹⁰+M_{WD}¹¹))/w̅ₘ

Above, w̅_{f} and w̅ₘ are the average female and male fitness:

w̅_{f}=F_{WW}+w_{WW}¹⁰F_{WW}¹⁰+w_{WW}⁰¹F_{WW}⁰¹+w_{WW}¹¹F_{WW}¹¹+w_{WD}¹⁰F_{WD}¹⁰+w_{WD}⁰¹F_{WD}⁰¹+w_{WD}¹¹F_{WD}¹¹+F_{WR}+F_{WR}¹⁰w_{WR}¹⁰+w_{WR}⁰¹F_{WR}⁰¹+w_{WR}¹¹F_{WR}¹¹

w̅ₘ=M_{WW}+M_{WW}¹⁰+M_{WW}⁰¹+M_{WW}¹¹+M_{WD}¹⁰+M_{WD}⁰¹+M_{WD}¹¹+M_{WR}+M_{WR}¹⁰+M_{WR}⁰¹+M_{WR}¹¹+M_{DD}+M_{DR}+M_{RR}=1

To model cage experiments, the inventors started with an equal number of males and females, with an initial frequency of wildtype females in the female population of F_WW=1, wildtype males in the male population of M_{WW}=1/2, and M_{WD}⁰¹=1/2 heterozygote drive males that inherited the drive from their fathers. Assuming a 50:50 ratio of males and females in progeny, after the starting generation, genotype frequencies of type i/j in the next generation (t+1) are the same in males and females, Fᵢⱼ(t+1)=Mᵢⱼ(t+1). Both are given by Gᵢⱼ(t+1) in the following set of equations in terms of the gamete proportions in the previous generation, assuming random mating:

G_{WW}(t+1)=e_{W}s_{W}

G_{WW}¹⁰(t+1)=e*_{W}s_{W}

G_{WW}⁰¹(t+1)=e_{W}s*_{W}

G_{WW}¹¹(t+1)=e*_{W}s*_{W}

G_{WD}¹⁰(t+1)=e*_{D}s_{W}

G_{WD}⁰¹(t+1)=e_{W}s*_{D}

G_{WD}¹¹(t+1)=e*_{W}s*_{D}+e*_{D}s*_{W}

G_{WR}(t+1)=e_{W}s_{R}+e_{R}s_{W}

G_{WR}¹⁰(t+1)=e*_{W}s_{R}+e*_{R}s_{W}

G_{WR}⁰¹(t+1)=e_{W}s*_{R}+e_{R}s*_{W}

G_{WR}¹¹(t+1)=e*_{W}s*_{R}+e*_{R}s*_{W}

G_{DD}(t+1)=e*_{D}s*_{D}

G_{DR}(t+1)=(e_{R}+e*_{R})s*_{D}+e*_{D}(s_{R}+s*_{R})

G_{RR}=(e_{R}+e*_{R})(s_{R}+s*_{R})

The frequency of transgenic individuals can be compared with experiment (fraction of RFP+ individuals):

f_{RFP+}=F_{WD}¹⁰+F_{WD}⁰¹+F_{WD}¹¹+F_{DD}+F_{DR}+M_{WD}¹⁰+M_{WD}⁰¹+M_{WD}¹¹+M_{DD}+M_{DR}

All calculations were carried out using Wolfram Mathematica²³.

PCR

The PCR reactions were performed using Phusion High Fidelity Master Mix. Initial denaturation was performed in 98° C. for 30 seconds. Primer annealing was performed at a temperature range of 60-72° C. for 30 seconds and elongation was performed at a temperature of 72° C. for 30 seconds per kb.

TABLE 2
Primers used in this study

| | |
|---|---|
| dsxgRNA-F | TGCTGTTTAACACAGGTCAAGCGG-SEQ ID No: 4 |
| dsxgRNA-R | AAACCCGCTTGACCTGTGTTAAAC-SEQ ID No: 5 |
| dsxΦ31L-F | GCTCGAATTAACCATTGTGGACCGGTCTTGTGTTTAGCAG |
| | GCAGGGGA-SEQ ID No: 6 |
| dsxΦ31L-R | TCCACCTCACCCATGGGACCCACGCGTGGTGCGGGTCACC |
| | GAGATGTTC-SEQ ID No: 7 |
| dsxΦ31R-F | CACCAAGACAGTTAACGTATCCGTTACCTTGACCTGTGTTA |
| | AACATAAAT-SEQ ID No: 8 |
| dsxΦ31R-R | GGTGGTAGTGCCACACAGAGAGCTTCGCGGTGGTCAACG |
| | AATACTCACG-SEQ ID No: 9 |
| zpgprCRISPR-F | GCTCGAATTAACCATTGTGGACCGGTCAGCGCTGGCGGTG |
| | GGGA-SEQ ID No: 10 |
| zpgprCRISPR-R | TCGTGGTCCTTATAGTCCATCTCGAGCTCGATGCTGTATTT |
| | GTTGT-SEQ ID No: 11 |
| zpgteCRISPR-F | AGGCAAAAAAGAAAAAGTAATTAATTAAGAGGACGGCGA |
| | GAAGTAATCAT-SEQ ID No: 12 |
| zpgteCRISPR-R | TTCAAGCGCACGCATACAAAGGCGCGCCTCGCATAATGAA |
| | CGAACCAAAGG-SEQ ID No: 13 |
| dsxin3-F | GGCCCTTCAACCCGAAGAAT-SEQ ID No: 14 |
| dsxex6-R | CTTTTTGTACAGCGGTACAC-SEQ ID No: 15 |
| GFP-F | GCCCTGAGCAAAGACCCCAA-SEQ ID No: 16 |
| dsxex4-F | GCACACCAGCGGATCGACGAAG-SEQ ID No: 17 |
| dsxex5-R | CCCACATACAAAGATACGGACAG-SEQ ID No: 18 |
| dsxex6-R | GAATTTGGTGTCAAGGTTCAGG-SEQ ID No: 19 |
| 3xP3 | TATACTCCGGCGGTCGAGGGTT-SEQ ID No: 20 |
| hCas9-F | CCAAGAGAGTGATCCTGGCCGA-SEQ ID No: 21 |
| dsxex5-R1 | CTTATCGGCATCAGTTGCGCAC-SEQ ID No: 22 |
| dsxin4-F | GGTGTTATGCCACGTTCACTGA-SEQ ID No: 23 |
| RFP-R | CAAGTGGGAGCGCGTGATGAAC-SEQ ID No: 24 |

TABLE 3
Primers used to amplify the promoters

| | |
|---|---|
| nos-pr-F | GTGAACTTCCATGGAATTACGT- |
| | SEQ ID No: 67 |
| nos-pr-R | CTTGCTTTCTAGAACAAAAGGATC- |
| | SEQ ID No: 68 |
| nos-ter-F | GACAGAGTCGTTCGTTCATT-SEQ |
| | ID No: 69 |
| nos-ter-R | GTAATTAGTGTTCATTTTAG-SEQ |
| | ID No: 70 |
| zpg-pr-F | CAGCGCTGGCGGTGGGGA-SEQ ID |
| | No: 71 |
| zpg-pr-R | CTCGATGCTGTATTTGTTGT-SEQ |
| | ID No: 72 |
| zpg-ter-F | GAGGACGGCGAGAAGTAATCAT- |
| | SEQ ID No: 73 |
| zpg-ter-R | TCGCATAATGAACGAACCAAAGG- |
| | SEQ ID No: 74 |
| exu-pr-F | GGAAGGTGATTGCGATTCCATGT- |
| | SEQ ID No: 75 |
| exu-pr-R | TTTGTACAAGCTACACAAGAGAAGG- |
| | SEQ ID No: 76 |
| exu-ter-F | GCGTGAGCCGGAGAAAGC-SEQ ID |
| | No: 77 |
| exu-ter-R | ACTGCTACTGTGCAACACATC-SEQ |
| | ID No: 78 |

TABLE 4
Primers used to assemble the vectors and verify the insertions

| | |
|---|---|
| nos-pr-CRISPR-F | GCTCGAATTAACCATTGTGGACCGGTGTGAACTTCCATGGAATTACGT-SEQ ID |
| | No: 79 |
| nos-pr-CRISPR-R | TCGTGGTCCTTATAGTCCATCTCGAGCTTGCTTTCTAGAACAAAAGGATC-SEQ |
| | ID No: 80 |
| nos-ter-CRISPR-F | GCCGGCCAGGCAAAAAAGAAAAAGTAATTAATTAAGACAGAGTCGTTCGTTCATT- |
| | SEQ ID No: 81 |
| nos-ter-CRISPR-r | TCAACCCTTCAAGCGCACGCATACAAAGGCGCGCCGTAATTAGTGTTCATTTTAG- |
| | SEQ ID No: 82 |
| zpg-pr-CRISPR-F | GCTCGAATTAACCATTGTGGACCGGTCAGCGCTGGCGGTGGGGA-SEQ ID No: |
| | 10 |
| zpg-pr-CRISPR-R | TCGTGGTCCTTATAGTCCATCTCGAGCTCGATGCTGTATTTGTTGT-SEQ ID |
| | No: 11 |
| zpg-ter-CRISPR-F | AGGCAAAAAAGAAAAAGTAATTAATTAAGAGGACGGCGAGAAGTAATCAT-SEQ |
| | ID No: 12 |
| zpg-ter-CRISPR-R | TTCAAGCGCACGCATACAAAGGCGCGCCTCGCATAATGAACGAACCAAAGG-SEQ |
| | ID No: 13 |
| exu-pr-CRISPR-F | GCTCGAATTAACCATTGTGGACCGGTGGAAGGTGATTGCGATTCCATGT-SEQ |
| | ID No: 83 |
| exu-pr-CRISPR-R | TCGTGGTCCTTATAGTCCATCTCGAGTTTGTACAAGCTACACAAGAGAAGG-SEQ |
| | ID No: 84 |
| exu-ter-CRISPR-F | AGGCAAAAAAGAAAAAGTAATTAATTAAGCGTGAGCCGGAGAAAGC-SEQ ID |
| | No: 85 |
| exu-ter-CRISPR-r | TTCAAGCGCACGCATACAAAGGCGCGCCACTGCTACTGTGCAACACATC-SEQ ID |
| | No: 86 |
| hCas9-F7 | CGGCGAACTGCAGAAGGGAA-SEQ ID No: 87 |
| RFP2qF | GTGCTGAAGGGCGAGATCCACA-SEQ ID No: 88 |
| Seq-7280-F | GCACAAATCCGATCGTGACA-SEQ ID No: 89 |
| Seq-7280-R | CAGTGGCAGTTCCGTAGAGA-SEQ ID No: 90 |

Results

To investigate whether dsx represented a suitable target for a gene drive approach aimed at suppressing population reproductive capacity, the inventors disrupted the intron 4-exon 5 boundary of dsx with the objective to prevent the formation of functional AgdsxF while leaving the AgdsxM transcript unaffected. The inventors injected A. gambiae embryos with a source of Cas9 and gRNA designed to selectively cleave the intron 4-exon 5 boundary in combination with a template for homology directed repair (HDR) to insert an eGFP transcription unit (FIG. 1c). Transformed individuals were intercrossed to generate homozygous and heterozygous mutants among the progeny. HDR-mediated integration was confirmed by a diagnostic PCR using primers that spanned the insertion site, producing a larger amplicon of the expected size for the HDR event and a smaller amplicon for the wild type allele, and thus allowing easy confirmation of genotypes (FIG. 1d).

The knock-in of the eGFP construct resulted in the complete disruption of the exon 5 (dsxF−) coding sequence and was confirmed by PCR and genomic sequencing of the chromosomal integration (FIG. 6). Crosses of heterozygote individuals produced, wild type, heterozygous and homozygous individuals for the dsxF− allele at the expected Mendelian ratio 1:2:1, indicating that there was no obvious lethality associated with the mutation during development (Table 4).

TABLE 4
Ratio of larvae recovered by intercrossing heterozygous dsx
ΦC31-knock-in mosquitoes

| GFP strong (dsxF^{-/-}) | GFP weak (dsxF^{-/+}) | no GFP (+/+) | Total |
|---|---|---|---|
| 262 (24.9%) | 523 (49.7%) | 268 (25.5%) | 1053 |

Larvae heterozygous for the exon 5 disruption developed into adult male and female mosquitoes with a sex ratio close to 1:1. On the contrary half of dsxF−/− individuals developed into normal males whereas the other half showed the presence of both male and female morphological features as well as a number of developmental anomalies in the internal and external reproductive organs (intersex).

To establish the sex genotype of these dsxF−/− intersex, the inventors introgressed the mutation into a line containing a Y-linked visible marker (RFP) and used the presence of this marker to unambiguously assign sex genotype among individuals heterozygous and homozygous for the null mutation. This approach revealed that the intersex phenotype was observed only in genotypic females that were homozygous for the null mutation. The inventors saw no effect in heterozygous mutants, suggesting that the female-specific isoform of dsx is haplosufficient.

Examination of external sexually dimorphic structures in dsxF−/− genotypic females showed several phenotypic abnormalities including: the development of dorsally rotated male claspers (and absent female cerci), longer flagellomeres associated with male-like plumose antennae (FIG. 2). The analysis of the internal reproductive organs of these individuals failed to reveal the presence of fully developed ovaries and spermathecae; instead they were replaced by male-accessory glands (MAGs) and in some cases (˜20%) by rudimentary pear-shaped organs resembling unstructured testes (FIG. 7).

Males carrying the dsxF− null mutation in heterozygosity or homozygosity showed wild type levels of fertility as measured by clutch size and larval hatching per mated female, as did heterozygous dsxF− female mosquitoes. On the contrary, intersex XX dsxF−/− female mosquitoes, though attracted to anaesthetised mice were unable to take a bloodmeal and failed to produce any eggs (FIG. 3).

The surprisingly drastic phenotype of dsxF−/− in females is proof of key functional role of exon 5 of dsx in the poorly understood sex differentiation pathway of A. gambiae mosquitoes and suggested that its sequence could represent a suitable target for gene drive approaches aimed at population suppression.

The inventors employed recombinase-mediated cassette exchange (RMCE) to replace the 3×P3::GFP transcription unit with a dsxFCRISPRh gene drive construct that consists of an RFP marker gene, a transcription unit to express the gRNA targeting dsxF, and the Cas9 gene under the control of the germline promoter of zero population growth (zpg) and its terminator sequence (FIG. 8). The zpg promoter has shown improved germline restriction of expression and specificity over the vasa promoter used in previous gene drive constructs (Hammond and Crisanti unpublished). Successful RMCE events that incorporated the dsxFCRISPRh into its target locus were confirmed in those individuals that had swapped the GFP for the RFP marker. During meiosis the Cas9/gRNA complex cleaves the wild type allele at the target sequence and the dsxFCRISPRh cassette is copied into wt locus via HDR (‘homing’), disrupting exon 5 in the process.

The ability of the dsxFCRISPRh construct to home and bypass Mendelian inheritance was analysed by scoring the rates of RFP inheritance in the progeny of heterozygous parents (referred to as dsxFCRISPRh/+ hereafter) crossed to wild type mosquitoes.

Surprisingly, high dsxFCRISPRh transmission rates of up to 100% were observed in the progeny of both heterozygous dsxFCRISPRh/+ male and female mosquitoes (FIG. 4a). The fertility of the dsxFCRISPRh line was also assessed to unravel potential negative effects due to ectopic expression of the nuclease in somatic cells and/or parental deposition of the nuclease into the newly fertilised embryos (FIG. 4b). These experiments showed that while heterozygous dsxFCRISPRh/+ males showed a fecundity rate (assessed as larval progeny per fertilised female) that did not differ from wild type males, heterozygous dsxFCRISPRh/+ female showed reduced fecundity overall (mean fecundity 49.8%+/−6.3% S.E., p<0.001).

Surprisingly, the inventors noticed a more severe reduction in the fertility of heterozygous females when the drive allele was inherited from their father (mean fecundity 21.7%+/−8.6%) rather than their mother (64.9%+/−6.9%) (FIG. 10). Without wishing to be bound to any particular theory, the inventors believe that this could be explained assuming a paternal deposition of active Cas9 nuclease into the newly fertilized zygote that stochastically induces conversion to of dsx to dsxF−, either through end-joining or HDR, in a significant number of cells resulting in a reduced fertility in females. Consistent with this hypothesis, some heterozygous females receiving a paternal dsxFCRISPRh allele showed a somatic mosaic phenotype that included, with varying penetrance, the absence of spermatheca and/or the formation of an incomplete clasper set. A mathematical model built considering the inheritance bias of the construct, the fecundity of heterozygous individuals, the phenotype of intersex as well as the paternal deposition of the nuclease on female fertility, indicated that the dsxFCRISPRh had the potential to reach 100% frequency in caged population in a span of 9-13 generations depending on starting frequency and stochasticity (FIG. 5a).

To test this hypothesis, caged wild type mosquito populations were mixed with individuals carrying the dsxFCRISPRh allele and subsequently monitored at each generation to assess the spread of the drive and quantify its effect on reproductive output. To mimic a hypothetical release scenario, the inventors started the experiment in two replicate cages putting together 300 wild type female mosquitoes with 150 wt male mosquitoes and 150 dsxFCRISPRh/+ male individuals and allowed them to mate. Eggs produced from the whole cage were counted and 650 eggs were randomly selected to seed the next generations. The larvae that hatched from the eggs were screened for the presence of the RFP marker to score the number of the progeny containing the dsxFCRISPRh allele in each generation. During the first three generations, the inventors observed in both caged populations an increase of the drive allele from 25% up to ˜69% and thereafter they diverged. In cage 2 the drive reached 100% frequency by generation 7; in the following generation no eggs were produced and the population collapsed. In cage 1 the drive allele reached 100% frequency at generation 11 after drifting around 65% for two generations. This cage population also failed to produce eggs in the next generation. Though the two cages showed some apparent differences in the dynamics of spreading both curves fall within the prediction of the model (FIG. 5b). A summary of the cage trials is shown in table 6.

The inventors also monitored at different generations the occurrence of mutations at the target site to identify the occurrence of nuclease resistant functional variants. Amplicon sequencing of the target sequence from pooled population samples collected at generation 2, 3, 4 and 5 revealed the presence of several low frequency indels generated at the cleavage site, none of which appeared to encode for a functional AgdsxF transcript (FIGS. 10A-C). Accordingly, none of the variants identified showed any signs of positive selection as the drive progressively increased in frequency over generations, thus indicating that the selected target sequence has rigid functional and structural constraints. This notion is supported by the high degree of conservation of exon 5 in A. gambiae mosquitoes^{16, 17} and the presence of highly regulated splice site critical for the mosquito reproductive biology.

Heterozygous and homozygous individuals for the dsxF⁻ allele were separated based on the intensity of fluorescence afforded by the GFP transcription unit within the knockout allele. Homozygous mutants were distinguishable as recovered in the expected Mendelian ratio of 1:2:1 suggesting that the disruption of the female-specific isoform of Agdsx is not lethal at the L1 larval stage.

TABLE 5
Genetic females homozygous for the insertion carry male-specific
characteristics

| | Genetic Males | | | Genetic Females | | |
|---|---|---|---|---|---|---|
| Characteristic | dsxF^{+/+} | dsxF^{+/-} | dsxF^{-/-} | dsxF^{+/+} | dsxF^{+/-} | dsxF^{-/-} |
| Pupal genital | male | male | male | female | female | male |
| lobe | | | | | | |
| Claspers | ✓ | ✓ | ✓ | x | x | ✓ |
| Cercus | x | x | x | ✓ | ✓ | x |
| Spermatheca | x | x | x | ✓ | ✓ | x |
| MAGs | ✓ | ✓ | ✓ | x | x | ✓ |
| Feed on blood | x | x | x | ✓ | ✓ | x |
| Can lay eggs | x | x | x | ✓ | ✓ | x |
| Plumose | ✓ | ✓ | ✓ | x | x | ✓ |
| antennae | | | | | | |
| Pilose antennae | x | x | x | ✓ | ✓ | x |

The inventors assume that parental effects on fitness (egg production and hatching rates) for non-drive (W/W, W/R) females with nuclease from one or both parents are the same as observed values for drive heterozygote (W/D) females with parental effects. For combined maternal and paternal effects (nuclease from both parents), the minimum of the observed values for maternal and paternal effect is assumed.

TABLE 6
Summary of values obtained from the cage trials

| | Cage Trial 1 | | | | Cage Trial 2 | | | |
|---|---|---|---|---|---|---|---|---|
| | Transgenic | Hatching | | Repr. | | | Egg | Repr. |
| | Rate | Rate | Egg Output | Load | Transgenic | Hatching | Output | Load |
| Generation | (%) | (%) | (N) | (%) | Rate (%) | Rate (%) | (N) | (%) |
| G0 | 25 | — | 27462 | — | 25 | — | 26895 | — |
| | (150/600) | | | | (150/600) | | | |
| G1 | 49.65 | 88.62 | 17405 | 36.62 | 50 | 86.15 | 16578 | 38.36 |
| | (268/576) | (576/650) | | | (280/560) | (560/650) | | |
| G2 | 62.01 | 74.92 | 14957 | 45.54 | 61.79 | 80.92 | 15565 | 42.13 |
| | (302/487) | (487/650) | | | (325/526) | (526/650) | | |
| G3 | 68.94 | 76.77 | 11249 | 59.04 | 68.05 | 74.15 | 9376 | 65.14 |
| | (344/499) | (499/650) | | | (328/482) | (482/650) | | |
| G4 | 67.67 | 71.85 | 9170 | 66.61 | 85.41 | 71.69 | 6514 | 75.78 |
| | (316/467) | (467/650) | | | (398/466) | (466/650) | | |
| G5 | 58.67 | 69.23 | 11364 | 58.62 | 86.5 | 61.54 | 4805 | 81.13 |
| | (264/450) | (450/650) | | | (346/400) | (400/650) | | |
| G6 | 63.3 | 70 | 7727 | 71.86 | 90.09 | 52.77 | 4210 | 84.35 |
| | (288/455) | (455/650) | | | (309/343) | (343/650) | | |
| G7 | 69.47 | 78.62 | 7785 | 71.65 | 100 | 55.85 | 1668 | 93.8 |
| | (355/511) | (511/650) | | | (363/363) | (363/650) | | |
| G8 | 70.07 | 70.92 | 6293 | 77.08 | 100 | 42.77 | 0 | 100 |
| | (323/461) | (461/650) | | | (278/278) | (278/650) | | |
| G9 | 75.58 | 66.15 | 4107 | 85.04 | — | — | — | — |
| | (325/430) | (430/650) | | | | | | |
| G10 | 95.71 | 57.38 | 4146 | 84.90 | | | | |
| | (357/373) | 373/650 | | | | | | |
| G11 | 100 | 57.54 | 2645 | 90.37 | | | | |
| | (374/253) | (374/650) | | | | | | |
| G12 | 100 | 38.92 | 0 | 100 | | | | |
| | (253/253) | (253/650) | | | | | | |

Transgenic rate, hatching rate, egg output and reproductive load at each generation during the cage experiment. The reproductive load indicates the suppression of egg production at each generation compared to the first generation.

Phenotypic assays were performed to measure simultaneously the fertility and transmission rates for each of three drives (FIG. 15c). To assess the level of homing, drive heterozygotes were crossed to wild-type, allowed to lay individually, and their progeny scored for the presence of DsRed linked to the construct (FIG. 15c).

Maternally or paternally deposited Cas9 can cause resistant mutations in the embryo that may reduce the rate of homing in the next generation (Hammond & Kyrou et al. 2017). To test this effect, the inventors separated male and female drive heterozygotes by whether they had inherited the drive from their mother or father and scored inheritance of the drive in their progeny (FIG. 15c). Irrespective of drive inheritance, all three promoters induced homing in males, while zpg-CRISPR^{h} and nos-CRISPR^{h} also showed biased transmission in females. Transmission rates for zpg-CRISPR^{h} exceeded 90.6% in males and 97.8% in females, falling only slightly below previously observed rates for vas2-CRISPR^{h} at 99.6% in males and 97.7% in females (Hammond et al. 2016). The nos promoter also showed high transmission at more than 83.6% in males and 85.1% in females. These rates were significantly higher when the drive was inherited from a male parent (99.1% in males and 99.6% in females) indicating that nos::Cas9 is maternally deposited. The exu promoter allowed rates of biased transmission in males (64%) and no bias in females (51%). These rates of homing remained similar after more than 20 generations, demonstrating that the drives are highly stable.

Fertility assays were performed to measure the larval output in individual crosses of drive heterozygotes to wild-type (FIG. 15c). All new drives showed a marked improvement in relative fertility when compared to wild-type control. Where vas-CRISPR^{h} females showed approximately 8.4% relative female fertility, the relative fertility of zpg-CRISPR^{h} (50-58.3%), nos-CRISPR^{h} (40.2-55.9%), and exu-CRISPR^{h} (75.5-77.4%) females were much improved. Moreover, a reduction in larval output of nos-CRISPR^{h} and exu-CRISPR^{h} males likely represents the stochastic variation brought about by different rearing and laying conditions rather than by nuclease activity itself.

Large differences between wild-type controls support this hypothesis. As such, the values above are used only as a rough estimate of fertility that serve to demonstrate the dramatic improvement over vas2.

To test the potential for zpg-CRISPR^{h} to spread throughout nave populations of malaria mosquitoes, two replicate cages were initiated with either 10% or 50% of drive heterozygotes, and monitored for 16 generations. Remarkably, the drive spread to more than 97% of the population in all four replicates (FIG. 16) and had achieved complete population modification in one of the two 50% release cages after just four generations. In all four releases, the drive sustained more than 95% frequency for at least 3 generations before its spread was reversed by the gradual selection of drive-resistant alleles. Notably, the inventors observed similar dynamics of spread whether released at 50% or 10%, demonstrating that initial release frequency has little impact upon the potential to spread. These results are all the more surprising when compared to vas2-driven CRISPR^{h} targeted to the exact same locus at AGAP007280. Here, the spread of the drive was slower and resistance arose before it reached 80% frequency in the population. (Hammond et al. 2016).

Resistant mutations arise when there is a change to the target site sequence that prevents further recognition or cleavage by the nuclease, but also encodes a gene product that can rescue against the sterile knock-out phenotype. Though these may be pre-existing in a population, they are overwhelmingly produced by the gene drive itself from error-prone non-homologous end-joining (NHEJ) or microhomology-mediated end-joining (MMEJ) in the small fraction of cleaved chromosomes that are not repaired by homing in the germline, or in the embryo following cleavage by maternally- or paternally-deposited nuclease (Hammond & Kyrou et al. 2017).

To investigate the nature and frequency of resistance in the zpg-CRISPRh release cages, the inventors performed amplicon sequencing across the target locus in samples of pooled individuals collected before, during and after the emergence of resistance at generations 0, 2, 5 and 8 (FIG. 17). In stark contrast to the vas2-based drives, use of the zpg promoter reduces both the creation and selection of resistant mutations. Throughout the entire caged experiment, the inventors identified only 2 mutant alleles present at more than 1% frequency amongst non-drive alleles and both were present in each of the zpg-CRISPRh cages. Both mutations were in-frame deletions of 3 bp (203-GAG—SEQ ID No: 65) or 6 bp (203-GAGGAG—SEQ ID No: 66) at the target site and had been previously confirmed to provide resistance to the vas2-based gene drive (Hammond and Kyrou et al. 2017). By generation 8, one of the two mutations had reached a frequency greater than 90% amongst non-drive alleles yet each cage had selected a different allele—suggesting that selection for one or the other resistant mutation is stochastic and not because one is more effective at restoring fertility. In contrast to this, vas2-CRISPRh generated between 6 and 12 mutant alleles above 1% frequency in each replicate of both early and late generations, and this high variance in mutant alleles was maintained over time despite a strong stratification towards those conferring resistance (Hammond and Kyrou et al. 2017).

### CONCLUSIONS

The regulatory sequences of zpg, nos and exu described herein offer a clear advantage over and above the current best system (i.e. the vasa2 promoter) used for germline nuclease expression in gene drives designed for the malaria mosquito, showing:

1) surprisingly high rates of biased transmission into the offspring of both male and female mosquitoes;

2) substantially reduced fitness cost;

3) reduced end-joining mutations that are the major cause of resistance to gene drive; and

4) vastly improved spread in caged experiments in terms of speed, persistence and maximum frequency of the drive.

Gene drives based upon these promoter sequences are far superior to all previously tested gene drives and could be used for both population replacement and population suppression strategies. The improvements in gene drive efficacy can be attributed to vast improvements in spatio-temporal regulation of Cas9 nuclease expression that is brought about by the use of these novel regulatory sequences, specifically an improvement in restriction to the germline.

To illustrate the magnitude of improvement, the inventors observed a relative fitness in females of more than 80% compared to only 7% using the vasa2 promoter, as shown in FIG. 15D. The ultimate goal of gene drive technology is to modify entire populations when starting from low initial release frequency. Using identical methods to previously published research, the inventors have observed the first ever spread to >99% of individuals in a caged population using the zpg promoter, compared to a maximum frequency of 80% in the previous best tested gene drive based upon the vasa2 promoter. The inventors have demonstrated this spread when releasing from 50% initial frequency (mirroring previous research) and also from 10% initial frequency that is more relevant to vector control. The improved activity can be attributed entirely to the use of improved germline promoters because the gene drives were otherwise identical and the observed improvements in spread are predicted by mathematical models based upon observed characteristics of the transgenic lines based upon these promoters.

The inventors have demonstrated that gene drives built using these promoters require no further improvement to invade entire mosquito populations and meet the requirements for a gene drive system aimed at population replacement. The regulatory sequences described herein may be used for a range of technologies currently under development, including improvements to mosquito transformation, driving endonuclease genes, and other gene drive technologies that rely upon expression in the mosquito germline.

### REFERENCES

1. Gantz, V. M. et al. Highly efficient Cas9-mediated gene drive for population modification of the malaria vector mosquito Anopheles stephensi. Proc Natl Acad Sci USA 112, E6736-6743 (2015).

2. Hammond, A. et al. A CRISPR-Cas9 gene drive system targeting female reproduction in the malaria mosquito vector Anopheles gambiae. Nat Biotechnol 34, 78-83 (2016).

3. Burt, A. Site-specific selfish genes as tools for the control and genetic engineering of natural populations. Proc Biol Sci 270, 921-928 (2003).

4. Deredec, A., Godfray, H. C. & Burt, A. Requirements for effective malaria control with homing endonuclease genes. Proc Natl Acad Sci USA 108, E874-880 (2011).

5. Hamilton, W. D. Extraordinary sex ratios. A sex-ratio theory for sex linkage and inbreeding has new implications in cytogenetics and entomology. Science 156, 477-488 (1967).

6. Galizi, R. et al. A synthetic sex ratio distortion system for the control of the human malaria mosquito. Nat Commun 5, 3977 (2014).

7. Magnusson, K. et al. Demasculinization of the Anopheles gambiae X chromosome. BMC Evol Biol 12, 69 (2012).

8. Champer, J. et al. Novel CRISPR/Cas9 gene drive constructs reveal insights into mechanisms of resistance allele formation and drive efficiency in genetically diverse populations. PLoS Genet 13, e1006796 (2017).

9. Hammond, A. M. et al. The creation and selection of mutations resistant to a gene drive over multiple generations in the malaria mosquito. PLoS Genet 13, e1007039 (2017).

10. Marshall, J. M., Buchman, A., Sanchez, C. H. & Akbari, O. S. Overcoming evolved resistance to population-suppressing homing-based gene drives. Sci Rep 7, 3776 (2017).

11. Unckless, R. L., Clark, A. G. & Messer, P. W. Evolution of Resistance Against CRISPR/Cas9 Gene Drive. Genetics 205, 827-841 (2017).

12. Burtis, K. C. & Baker, B. S. Drosophila doublesex gene controls somatic sexual differentiation by producing alternatively spliced mRNAs encoding related sex-specific polypeptides. Cell 56, 997-1010 (1989).

13. Graham, P., Penn, J. K. & Schedl, P. Masters change, slaves remain. Bioessays 25, 1-4 (2003).

14. Krzywinska, E., Dennison, N. J., Lycett, G. J. & Krzywinski, J. A maleness gene in the malaria mosquito Anopheles gambiae. Science 353, 67-69 (2016).

15. Scali, C., Catteruccia, F., Li, Q. & Crisanti, A. Identification of sex-specific transcripts of the Anopheles gambiae doublesex gene. J Exp Biol 208, 3701-3709 (2005).

16. Neafsey, D. E. et al. Mosquito genomics. Highly evolvable malaria vectors: the genomes of 16 Anopheles mosquitoes. Science 347, 1258522 (2015).

17. Anopheles gambiae Genomes, C. et al. Genetic diversity of the African malaria vector Anopheles gambiae. Nature 552, 96-100 (2017).

18. Murray, S. M., Yang, S. Y. & Van Doren, M. Germ cell sex determination: a collaboration between soma and germline. Curr Opin Cell Biol 22, 722-729 (2010).

19. Curtis, C. F. Possible use of translocations to fix desirable genes in insect pest populations. Nature 218, 368-369 (1968).

20. National Academies of Sciences, E. & Medicine Gene Drives on the Horizon: Advancing Science, Navigating Uncertainty, and Aligning Research with Public Values. (The National Academies Press, Washington, D.C.; 2016).

21. Papathanos, P. A., Windbichler, N., Menichelli, M., Burt, A. and Crisanti, A. The vasa regulatory region mediates germline expression and maternal transmission of proteins in the malaria mosquito Anopheles gambiae: a versatile tool for genetic control strategies. BMC Mol Biol 10, 65, (2009).

22. Hammond, A. M. et al. The creation and selection of mutations resistant to a gene drive over multiple generations in the malaria mosquito. PLoS Genet 13, e1007039 (2017).

23. Wolfram Research, Inc., 2017 Mathematica 11.2, Champaign, Ill.

1-20. (canceled)

21. A gene drive genetic construct comprising a polynucleotide comprising:
a. a nucleic acid sequence substantially as set out in any one of SEQ ID NO: 1, 2 or 3, or a variant or fragment thereof having at least 50% sequence identity with SEQ ID NO: 1, 2 or 3;
b. an expression cassette comprising the polynucleotide of (a); or
c. a recombinant vector comprising the polynucleotide of (a) or the expression cassette of (b).

22. The gene drive genetic construct according to claim 21, wherein the polynucleotide sequence substantially restricts the activity of the gene drive genetic construct for germline expression of the construct in an arthropod.

23. The gene drive genetic construct according to claim 22, wherein the arthropod is an insect, optionally wherein the insect is a mosquito.

24. The gene drive genetic construct according to claim 22 wherein the arthropod is a mosquito and wherein the mosquito is of the subfamily Anophelinae, optionally wherein the mosquito is selected from a group consisting of: Anopheles gambiae; Anopheles coluzzi; Anopheles menus; Anopheles arabiensis; Anopheles quadriannulatus; Anopheles stephensi; Anopheles funestus; and Anopheles melas.

25. A method of producing a genetically modified host cell or arthropod comprising;
(a) introducing, into a host cell, an expression cassette comprising a polynucleotide comprising a nucleic acid sequence substantially as set out in any one of SEQ ID No: 1, 2 or 3, or a variant or fragment thereof having at least 50% sequence identity with SEQ ID No: 1, 2 or 3, operably linked to a transgene; or
(b) introducing, into an arthropod gene, a gene drive genetic construct comprising:
(i) a polynucleotide comprising a nucleic acid sequence substantially as set out in any one of SEQ ID No: 1, 2 or 3, or a variant or fragment thereof having at least 50% sequence identity with SEQ ID No: 1, 2 or 3;
(ii) an expression cassette comprising the polynucleotide of (i); or
(iii) a recombinant vector comprising the polynucleotide of (i) or the expression cassette of (ii).

26-33. (canceled)

34. An expression cassette comprising a polynucleotide comprising a nucleic acid sequence substantially as set out in any one of SEQ ID No: 1, 2 or 3, or a variant or fragment thereof having at least 50% sequence identity with SEQ ID No: 1, 2 or 3, operably linked to a transgene.

35. The expression cassette according to claim 34, wherein the transgene is selected from the group consisting of: a CRISPR nuclease, a Zinc finger nuclease, a TALEN-derived nuclease, Cre recombinase, a piggyback transposase; and a φC31 integrase.

36. The expression cassette according to either claim 34, wherein the transgene is a CRISPR nuclease, optionally wherein the transgene is Cpf1 or Cas9.

37. An expression cassette according to claim 34, wherein the polynucleotide comprises or consists of a nucleic acid sequence having at least 80% or 90% sequence identity with SEQ ID No: 1, or a variant or fragment thereof.

38. An expression cassette according to claim 34, wherein the polynucleotide comprises or consists of a nucleic acid sequence having at least 95% or 99% sequence identity with SEQ ID No: 1, or a variant or fragment thereof.

39. An expression cassette according to claim 34, wherein the polynucleotide sequence comprises or consists of a nucleic acid sequence having at least 80% or 90% sequence identity with SEQ ID No: 2, or a variant or fragment thereof.

40. An expression cassette according to claim 34, wherein the polynucleotide sequence comprises or consists of a nucleic acid sequence having at least 95% or 99% sequence identity with SEQ ID No: 2, or a variant or fragment thereof.

41. An expression cassette according to claim 34, wherein the polynucleotide sequence comprises or consists of a nucleic acid sequence having at least 80% or 90% sequence identity with SEQ ID No: 3, or a variant or fragment thereof.

42. An expression cassette according to claim 34, wherein the polynucleotide sequence comprises or consists of a nucleic acid sequence having at least 95% or 99% sequence identity with SEQ ID No: 3, or a variant or fragment thereof.

43. The expression cassette according to claim 34, wherein the polynucleotide initiates gene expression of a coding sequence operatively connected thereto in the germline cells only.

44. The expression cassette according to claim 34, wherein the polynucleotide sequence is a promoter sequence that is substantially operative in only germline cells of an arthropod, optionally wherein the polynucleotide is a promoter sequence which is substantially operative in the male and female mosquito gonad cells at the time of meiosis.


SEQUENCE LISTING (90 sequences)

SEQ ID NO: 1 - DNA, 1074 bp, Anopheles gambiae
cagcgctggcggtggggacagctccggctgtggctgttcttgcgagtcctcttcctgcggcacatccctctcgtcgaccagttcagtttgctgagcgtaagcctgctgctgttcgtcctgcatcatcgggaccatttgtatgggccatccgccaccaccaccatcaccaccgccgtccatttctaggggcatacccatcagcatctccgcgggcgccattggcggtggtgccaaggtgccattcgtttgttgctgaaagcaaaagaaagcaaattagtgttgtttctgctgcacacgataattttcgtttcttgccgctagacacaaacaacactgcatctggagggagaaatttgacgcctagctgtataacttacctcaaagttattgtccatcgtggtataatggacctaccgagcccggttacactacacaaagcaagattatgcgacaaaatcacagcgaaaactagtaattttcatctatcgaaagcggccgagcagagagttgtttggtattgcaacttgacattctgctgcgggataaaccgcgacgggctaccatggcgcacctgtcagatggctgtcaaatttggcccggtttgcgatatggagtgggtgaaattatatcccactcgctgatcgtgaaaatagacacctgaaaacaataattgttgtgttaattttacattttgaagaacagcacaagttttgctgacaatatttaattacgtttcgttatcaacggcacggaaagattatctcgctgattatccctctcgctctctctgtctatcatgtcctggtcgttctcgcgtcaccccggataatcgagagacgccatttttaatttgaactactacaccgacaagcatgccgtgagctctttcaagttcttctgtccgaccaaagaaacagagaataccgcccggacagtgcccggagtgatcgatccatagaaaatcgcccatcatgtgccactgaggcgaaccggcgtagcttgttccgaatttccaagtgcttccccgtaacatccgcatataacaaacagcccaacaacaaatacagcatcgag

SEQ ID NO: 2 - DNA, 2092 bp, Anopheles gambiae
gtgaacttccatggaattacgtgctttttcggaatggagttgggctggtgaaaaacacctatcagcaccgcacttttcccccggcatttcaggttatacgcagagacagagactaaatattcacccattcatcacgcactaacttcgcaatagattgatattccaaaactttcttcacctttgccgagttggattctggattctgagactgtaaaaagtcgtacgagctatcatagggtgtaaaacggaaaacaaacaaacgtttaatggactgctccaactgtaatcgcttcacgcaaacaaacacacacgcgctgggagcgttcctggcgtcacctttgcacgatgaaaactgtagcaaaactcgcacgaccgaaggctctccgtccctgctggtgtgtgtttttttcttttctgcagcaaaattagaaaacatcatcatttgacgaaaacgtcaactgcgcgagcagagtgaccagaaataccgatgtatctgtatagtagaacgtcggttatccgggggcggattaaccgtgcgcacaaccagttttttgtgcagctttgtagtgtctagtggtattttcgaaattcatttttgttcattaacagttgttaaacctatagttattgattaaaataatattctactaacgattaaccgatggattcaaagtgaataaattatgaaactagtgatttttttaaatttttatatgaatttgacatttcttggaccattatcatcttggtctcgagctgcccgaataatcgacgttctactgtattcctaccgattttttatatgcctaccgacacacaggtgggccccctaaaactaccgatttttaatttatcctaccgaaaatcacagattgtttcataatacagaccaaaaagtcatgtaaccatttcccaaatcacttaatgtattaaactccatatggaaatcgctagcaaccagaaccagaagttcaacagagacaaccaatttccgtgtatgtacttcatgagatgagattggacgcgctggtaaaattttatatgggatttgacagataatgtaaggcgtgcgatttttttcatacgatggaatcaattcaagagtcaattgtgcaggatttatagaaacaatctcttatttatgttttgttatcgttacagttacagccctgtcctaagcggccgcgtgaaggcccaaaaaaaagggagtccccaacgctcagtagcaaatgtgcttctctatcattcgttgggttagaaaagcctcatgtgacttctatgaacaaaatctaaactatctcctttaaatagagaatggatgtattttttcgtgccactgaactttcgttgggaagattagatacctctccctccccccccctccctttcaacacttcaaaacctaccgaaaactaccgatacaatttgatgtacctaccgaagaccgccaaaataatctggccacactggctagatctgatgttttgaaacatcgccaaattttactaaataatgcacttgcgcgttggtgaagctgcacttaaacagattagttgaattacgctttctgaaatgtttttattaaacacttgttttttttaatacttcaatttaaagctacttcttggaatgataattctacccaaaaccaaaaccactttacaaagagtgtgtggttggtgatcgcgccggctactgcgacctgtggtcatcgctcatctcacgcacacatacgcacacatctgtcatttgaaaagctgcacacaatcgtgtgttgtgcaaaaaaccgttcgcgcacaaacagttcgcacatgtttgcaagccgtgcagcaaagggcttttgatggtgatccgcagtgtttggtcagctttttaatgtgttttcgcttaatcgcttttgtttgtgtaatgttttgtcggaataatttttatgcgtcgttacaaatgaaatgtacaatcctgcgatgctagtgtaaaacattgctaattcccggtaagaacgttcattacgctcggatatcatcttacgaagcgtgtgtatgtgcgctagtacattgacctttaaagtgatccttttgttctagaaagcaag

SEQ ID NO: 3 - DNA, 849 bp, Anopheles gambiae
ggaaggtgattgcgattccatgttgatgccaatatatgatgattttgttgcatattaatagttgttgttatgttttattcaaatttcaaagataatttactttacattacagttagtgagcatattatctactacataaacacatagatcaaactggtttacataaattcaaaaagtttggattaaaatcgcagcaattggttatgaaaaaatatgtgcataacgtaaatatcaagtaaatttttgcattgcatatttatagactcctgttacaatttcggaaaaatgaaaaatgttaattaatcaaagaagaaaaaacaaagaaattaaatcattaggtagcacaaccacaagtacatatttttatggcatgaatattcctctacactaacatattttatagcaattctattgatcgccttagtatagcggaattaccagaacggcactatagttgtctctgtttggcacacgcaatcatttttcatcccagggttgccatagcagtttggcgacggtcacgtagcatgcgaaggatttcgttcgcacaggatcacttttattctaacgtttgaagaaggcacatctcagtgcaagcgctctggaagctgcttttaccgaacgaactaacttttcaagtaacctcaaaaacttgtctctaacgacaccacgtgctatccgcgagtttcatttcccgtgcaaagttccccgatttagctatcattcgtgaacatttcgtagtgcctctaccctcaggtaagaccattcgaggtttaccaagttttgtgcaaagaacgtgcacagtaattttcgttctggtgaaaccttctcttgtgtagcttgtacaaa

SEQ ID NO: 4 - DNA, 24 bp, Artificial Sequence
  dsxgRNA-F primer
tgctgtttaacacaggtcaagcgg

SEQ ID NO: 5 - DNA, 24 bp, Artificial Sequence
  dsxgRNA-R primer
aaacccgcttgacctgtgttaaac

SEQ ID NO: 6 - DNA, 48 bp, Artificial Sequence
  dsx?31L-F
gctcgaattaaccattgtggaccggtcttgtgtttagcaggcagggga

SEQ ID NO: 7 - DNA, 49 bp, Artificial Sequence
  dsx?31L-R primer
tccacctcacccatgggacccacgcgtggtgcgggtcaccgagatgttc

SEQ ID NO: 8 - DNA, 50 bp, Artificial Sequence
  dsx?31R-F primer
caccaagacagttaacgtatccgttaccttgacctgtgttaaacataaat

SEQ ID NO: 9 - DNA, 49 bp, Artificial Sequence
  dsx?31R-R primer
ggtggtagtgccacacagagagcttcgcggtggtcaacgaatactcacg

SEQ ID NO: 10 - DNA, 44 bp, Artificial Sequence
  zpgprCRISPR-F primer
gctcgaattaaccattgtggaccggtcagcgctggcggtgggga

SEQ ID NO: 11 - DNA, 46 bp, Artificial Sequence
  zpgprCRISPR-R primer
tcgtggtccttatagtccatctcgagctcgatgctgtatttgttgt

SEQ ID NO: 12 - DNA, 50 bp, Artificial Sequence
  zpgteCRISPR-F primer
aggcaaaaaagaaaaagtaattaattaagaggacggcgagaagtaatcat

SEQ ID NO: 13 - DNA, 51 bp, Artificial Sequence
  zpgteCRISPR-R primer
ttcaagcgcacgcatacaaaggcgcgcctcgcataatgaacgaaccaaagg

SEQ ID NO: 14 - DNA, 20 bp, Artificial Sequence
  dsxin3-F primer
ggcccttcaacccgaagaat

SEQ ID NO: 15 - DNA, 20 bp, Artificial Sequence
  dsxex6-R
ctttttgtacagcggtacac

SEQ ID NO: 16 - DNA, 20 bp, Artificial Sequence
  GFP-F primer
gccctgagcaaagaccccaa

SEQ ID NO: 17 - DNA, 22 bp, Artificial Sequence
  dsxex4-F primer
gcacaccagcggatcgacgaag

SEQ ID NO: 18 - DNA, 23 bp, Artificial Sequence
  dsxex5-R primer
cccacatacaaagatacggacag

SEQ ID NO: 19 - DNA, 22 bp, Artificial Sequence
  dsxex6-R primer
gaatttggtgtcaaggttcagg

SEQ ID NO: 20 - DNA, 22 bp, Artificial Sequence
  3xP3 primer
tatactccggcggtcgagggtt

SEQ ID NO: 21 - DNA, 22 bp, Artificial Sequence
  hCas9-F
ccaagagagtgatcctggccga

SEQ ID NO: 22 - DNA, 22 bp, Artificial Sequence
  dsxex5-R1primer
cttatcggcatcagttgcgcac

SEQ ID NO: 23 - DNA, 22 bp, Artificial Sequence
  dsxin4-F primer
ggtgttatgccacgttcactga

SEQ ID NO: 24 - DNA, 22 bp, Artificial Sequence
  RFP-R primer
caagtgggagcgcgtgatgaac

SEQ ID NO: 25 - DNA, 30 bp, Artificial Sequence
  intron 4 exon 4 boundary 1
ttatgtttaacacaggtcaagcggtggtca

SEQ ID NO: 26 - DNA, 30 bp, Artificial Sequence
  intron 4 exon 5 boundary 2
aatacaaattgtgtccagttcgccaccagt

SEQ ID NO: 27 - DNA, 54 bp, Artificial Sequence
  dsx intron 4-exon 5 boundary in 6 species
cctttccattcatttatgtttaacacaggtcaagcggtggtcaacgaatactca

SEQ ID NO: 28 - DNA, 37 bp, Artificial Sequence
  intron 4 exon 5 boundary
gtttaacacaggtcaagcggtggtcaacgaatactca

SEQ ID NO: 29 - DNA, 26 bp, Artificial Sequence
  intron 4 exon 5 boundary
gtttaacacaggtcaacgaatactca

SEQ ID NO: 30 - DNA, 33 bp, Artificial Sequence
  intron 4 exon 5 boundary
gtttaacacaggtcggtggtcaacgaatactca

SEQ ID NO: 31 - DNA, 28 bp, Artificial Sequence
  intron 4 exon 5 boundary
gtttaacacggtggtcaacgaatactca

SEQ ID NO: 32 - DNA, 26 bp, Artificial Sequence
  intron 4 exon 5 boundary
gtttaacggtggtcaacgaatactca

SEQ ID NO: 33 - DNA, 36 bp, Artificial Sequence
  intron 4 exon 5 boundary
gtttaacacaggtcaacggtggtcaacgaatactca

SEQ ID NO: 34 - DNA, 34 bp, Artificial Sequence
  intron 4 exon 5 boundary
gtttaacacaggtccggtggtcaacgaatactca

SEQ ID NO: 35 - DNA, 29 bp, Artificial Sequence
  intron 4 exon 5 boundary
gtttaacaccggtggtcaacgaatactca

SEQ ID NO: 36 - DNA, 27 bp, Artificial Sequence
  intron 4 exon 5 boundary
gtttaaccggtggtcaacgaatactca

SEQ ID NO: 37 - DNA, 39 bp, Artificial Sequence
  intron 4 exon 5 boundary
gtttaacacaggtcataagcggtggtcaacgaatactca

SEQ ID NO: 38 - DNA, 39 bp, Artificial Sequence
  intron 4 exon 5 boundary
gtttaacacaggtcaaggacggtggtcaacgaatactca

SEQ ID NO: 39 - DNA, 129 bp, Anopheles gambiae
cctttccattcatttatgtttaacacaggtcaagcggtggtcaacgaatactcacgattgcataatctgaacatgtttgatggcgtggagttgcgcaataccacccgtcagagtggatgataaactttc

SEQ ID NO: 40 - DNA, 129 bp, Unknown; identical to SEQ ID NO: 39
  dsx intron 4-exon 5 boundary

SEQ ID NO: 41 - DNA, 129 bp, Unknown; identical to SEQ ID NO: 39
  dsx intron 4-exon 5 boundary

SEQ ID NO: 42 - DNA, 129 bp, Unknown; identical to SEQ ID NO: 39
  dsx intron 4-exon 5 boundary

SEQ ID NO: 43 - DNA, 129 bp, Unknown; identical to SEQ ID NO: 39
  dsx intron 4-exon 5 boundary

SEQ ID NO: 44 - DNA, 129 bp, Unknown
  dsx intron 4-exon 5 boundary
cctttccattcatttatgtttaacacaggtcaagcggtggtcaacgaatactcacgattgcataatctgaacatgtttgatggcgtggagttgcgtaataccacccgtcagagtggatgataaactttc

SEQ ID NO: 45 - DNA, 129 bp, Unknown
  dsx intron 4-exon 5 boundary
cctttccattcatttatgtttaacacaggtcaagcggtggtcaacgaatactcacgattgcataatctgaacatgttcgatggcgtggagttgcgcaataccacccgtcagagtggatgataaactttc

SEQ ID NO: 46 - DNA, 129 bp, Unknown
  dsx intron 4-exon 5 boundary
cctttccattcatttatgttcaacacaggtcaagcggtggtcaacgaatactcacgattgcataatctgaacatgttcgatggcgtggagttgcgcaataccacccgtcagagtggatgataaactttc

SEQ ID NO: 47 - DNA, 129 bp, Unknown
  dsx intron 4-exon 5 boundary
cctttccattcatttatgttcaacacaggtcaaacggtggtcaacgaatactcacgattgcataatctgaacatgttcgatggcgtggagttacgcaataccacccgtcagagtggatgataaactttc

SEQ ID NO: 48 - DNA, 128 bp, Unknown
  dsx intron 4-exon 5 boundary
cctttccattcatttatgttcaacacaggtcaaacggtggtcaacgaatactcacgattgcataatctgaacatgttcgatggcgtggagttacgcaataccacccgtcagagtggatgataaacttt

SEQ ID NO: 49 - DNA, 129 bp, Unknown
  dsx intron 4-exon 5 boundary
cctttccattcatttatgttcaacacaggtcaagcggtggtcaacgaatactcaagattgcataatctgaacatgttcgatggcgtggagttacgcaataccacccgtcagagtggatgataaactttc

SEQ ID NO: 50 - DNA, 129 bp, Unknown
  dsx intron 4-exon 5 boundary
cctttccattcatttatgttcaacacaggtcaagcggtggtcaacgaatactcacgattgcataatctgaacatgttcgatggcgtggagttacgcaataccacccgtcagagtggatgataaactttc

SEQ ID NO: 51 - DNA, 129 bp, Unknown
  dsx intron 4-exon 5 boundary
ccttaccatgcatttatgttcaacacaggtcaagcggtggtcaacgaatactcacgattgcataatctgaacatgttcgatggcgtggagttacgcaacaccacccgtcagagtggatgataaactttc

SEQ ID NO: 52 - DNA, 129 bp, Unknown
  dsx intron 4-exon 5 boundary
cctttccattcatttatgttcaacacaggtcaagcggtggtcaacgaatactcacgattgcataatctgaacatgttcgatggcgcggagttgcgcaataccacccgtcagagtggatgataaactttc

SEQ ID NO: 53 - DNA, 129 bp, Unknown; identical to SEQ ID NO: 52
  dsx intron 4-exon 5 boundary

SEQ ID NO: 54 - DNA, 129 bp, Unknown
  dsx intron 4-exon 5 boundary
cctttccattcatttatgctcaacacaggtcaggccgtggtcaacgaatactcacgattgcacaatctgaacatgttcgatggcgtggagttgcgcaacaccacccgtcagagtggatgataaactttc

SEQ ID NO: 55 - DNA, 129 bp, Unknown; identical to SEQ ID NO: 54
  dsx intron 4-exon 5 boundary

SEQ ID NO: 56 - DNA, 129 bp, Unknown
  dsx intron 4-exon 5 boundary
ctttgccatttatttatgcccaacacaggtcaggccgtggtcaacgaatactcacgattgcacaatctgaacatgttcgatggcgtagagttgcgcaacgccacccgccagagcggatgataaacttcc

SEQ ID NO: 57 - DNA, 37 bp, Unknown
  AGAP007280, and its target site in exon 6
cgaggtgaggaagaaagtgaggaggagggtggtagtg

SEQ ID NO: 58 - DNA, 37 bp, Unknown
  AGAP007280, and its target site in exon 6
gctccactccttctttcactcctcctcccaccatcac

SEQ ID NO: 59 - DNA, 129 bp, Unknown
  dsx female-specific Exon 5 with SNP variants
cctttccattcatttatgtttaacacaggtcaagcagtggtcaacgaatattcacgattgcataatctgaacatgtttgatggcgtggagttgcgcaataccacccgtcagagtggatgataaactttc

SEQ ID NO: 60 - DNA, 23 bp, Unknown
  wild type (WT) target site in dsx exon 5
gtttaacacaggtcaagcggtgg

SEQ ID NO: 61 - DNA, 23 bp, Unknown
  target site in dsx exon 5 containing the single SNP found in wild caught populations
gtttaacacaggtcaagcagtgg

SEQ ID NO: 62 - DNA, 20 bp, Unknown
  no PAM′ control
gtttaacacaggtcaagcgg

SEQ ID NO: 63 - DNA, 53 bp, Artificial Sequence
  Primer
tcgtcggcagcgtcagatgtgtataagagacagggagaaggtaaatgcgccac

SEQ ID NO: 64 - DNA, 54 bp, Artificial Sequence
  Primers
gtctcgtgggctcggagatgtgtataagagacaggcgcttctacactcgcttct

SEQ ID NO: 65 - DNA, 3 bp, Artificial Sequence
  resistance mutationat target site
gag

SEQ ID NO: 66 - DNA, 6 bp, Artificial Sequence
  resistance mutation at target site
gaggag

SEQ ID NO: 67 - DNA, 22 bp, Artificial Sequence
  nos-pr-F primer
gtgaacttccatggaattacgt

SEQ ID NO: 68 - DNA, 24 bp, Artificial Sequence
  nos-pr-R primer
cttgctttctagaacaaaaggatc

SEQ ID NO: 69 - DNA, 20 bp, Artificial Sequence
  nos-ter-F primer
gacagagtcgttcgttcatt

SEQ ID NO: 70 - DNA, 20 bp, Artificial Sequence
  nos-ter-R primer
gtaattagtgttcattttag

SEQ ID NO: 71 - DNA, 18 bp, Artificial Sequence
  zpg-pr-F primer
cagcgctggcggtgggga

SEQ ID NO: 72 - DNA, 20 bp, Artificial Sequence
  zpg-pr-R primer
ctcgatgctgtatttgttgt

SEQ ID NO: 73 - DNA, 22 bp, Artificial Sequence
  zpg-ter-F
gaggacggcgagaagtaatcat

SEQ ID NO: 74 - DNA, 23 bp, Artificial Sequence
  zpg-ter-R primer
tcgcataatgaacgaaccaaagg

SEQ ID NO: 75 - DNA, 23 bp, Artificial Sequence
  exu-pr-F primer
ggaaggtgattgcgattccatgt

SEQ ID NO: 76 - DNA, 25 bp, Artificial Sequence
  exu-pr-R primer
tttgtacaagctacacaagagaagg

SEQ ID NO: 77 - DNA, 18 bp, Artificial Sequence
  exu-ter-F
gcgtgagccggagaaagc

SEQ ID NO: 78 - DNA, 21 bp, Artificial Sequence
  exu-ter-R primer
actgctactgtgcaacacatc

SEQ ID NO: 79 - DNA, 48 bp, Artificial Sequence
  nos-pr-CRISPR-F primer
gctcgaattaaccattgtggaccggtgtgaacttccatggaattacgt

SEQ ID NO: 80 - DNA, 50 bp, Artificial Sequence
  nos-pr-CRISPR-R primer
tcgtggtccttatagtccatctcgagcttgctttctagaacaaaaggatc

SEQ ID NO: 81 - DNA, 55 bp, Artificial Sequence
  nos-ter-CRISPR-F primer
gccggccaggcaaaaaagaaaaagtaattaattaagacagagtcgttcgttcatt

SEQ ID NO: 82 - DNA, 55 bp, Artificial Sequence
  nos-ter-CRISPR-r primer
tcaacccttcaagcgcacgcatacaaaggcgcgccgtaattagtgttcattttag

SEQ ID NO: 83 - DNA, 49 bp, Artificial Sequence
  exu-pr-CRISPR-F primer
gctcgaattaaccattgtggaccggtggaaggtgattgcgattccatgt

SEQ ID NO: 84 - DNA, 51 bp, Artificial Sequence
  exu-pr-CRISPR-R primer
tcgtggtccttatagtccatctcgagtttgtacaagctacacaagagaagg

SEQ ID NO: 85 - DNA, 46 bp, Artificial Sequence
  exu-ter-CRISPR-F primer
aggcaaaaaagaaaaagtaattaattaagcgtgagccggagaaagc

SEQ ID NO: 86 - DNA, 49 bp, Artificial Sequence
  exu-ter-CRISPR-r primer
ttcaagcgcacgcatacaaaggcgcgccactgctactgtgcaacacatc

SEQ ID NO: 87 - DNA, 20 bp, Artificial Sequence
  hCas9-F7
cggcgaactgcagaagggaa

SEQ ID NO: 88 - DNA, 22 bp, Artificial Sequence
  RFP2qF
gtgctgaagggcgagatccaca

SEQ ID NO: 89 - DNA, 20 bp, Artificial Sequence
  Seq-7280-F
gcacaaatccgatcgtgaca

SEQ ID NO: 90 - DNA, 20 bp, Artificial Sequence
  Seq-7280-R
cagtggcagttccgtagaga